SEQUENCE LISTING (31 sequences)

SEQ ID NO: 1 - DNA, 363 bp, MOUSE SP.
cagatccagttggtgcagtctggacctgagctgaagaagcctggagagacagtcaggatctcctgcaaggcctctgggtatatcttcacaattgcgggaatacagtgggtgcaaaagatgccaggaaggggtttgaggtggattggatggataaacacccattctggagtgccagaatatgcagaagagttcaagggacggtttgccttttctttggaaacctctgccagaactgcatatttacagataagcaacctcaaggatgaggacacggctacgtatttctgtgcgcggatctactatggtaacaacgggggtgttatggactattggggtcaaggaacctcagtcaccgtctcctca

SEQ ID NO: 2 - protein, 121 aa, MOUSE SP.
QIQLVQSGPELKKPGETVRISCKASGYIFTIAGIQWVQKMPGRGLRWIGWINTHSGVPEYAEEFKGRFAFSLETSARTAYLQISNLKDEDTATYFCARIYYGNNGGVMDYWGQGTSVTVSS

SEQ ID NO: 3 - DNA, 321 bp, MOUSE SP.
gacatccagatgacccagtctccatcctccttatctgcctctctgggagaaagagtcagtctcacttgtcgggcaagtcaggaaattagtgattacttaacctggcttcagcagaaaccagatggaactattaaacgcctgatctacgtcgcatccagtttagattctggtgtcccaaaaaggttcagtggcagtaggtctgggtcagattattctctcaccatcagcagccttgagtctgaagattttgcagactattactgtctacaatatgctaattatccgtggacgttcggtggaggcaccaagctggaaatcaga

SEQ ID NO: 4 - protein, 107 aa, MOUSE SP.
DIQMTQSPSSLSASLGERVSLTCRASQEISDYLTWLQQKPDGTIKRLIYVASSLDSGVPKRFSGSRSGSDYSLTISSLESEDFADYYCLQYANYPWTFGGGTKLEIR

SEQ ID NO: 5 - DNA, 1353 bp, ARTIFICIAL SEQUENCE
  MOUSE HUMAN CHIMERA SEQUENCE
cagatccagttggtgcagtctggacctgagctgaagaagcctggagagacagtcaggatctcctgcaaggcctctgggtatatcttcacaattgcgggaatacagtgggtgcaaaagatgccaggaaggggtttgaggtggattggatggataaacacccattctggagtgccagaatatgcagaagagttcaagggacggtttgccttttctttggaaacctctgccagaactgcatatttacagataagcaacctcaaggatgaggacacggctacgtatttctgtgcgcggatctactatggtaacaacgggggtgttatggactattggggtcaaggaacctcagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 6 - protein, 451 aa, ARTIFICIAL SEQUENCE
  MOUSE HUMAN CHIMERA SEQUENCE
QIQLVQSGPELKKPGETVRISCKASGYIFTIAGIQWVQKMPGRGLRWIGWINTHSGVPEYAEEFKGRFAFSLETSARTAYLQISNLKDEDTATYFCARIYYGNNGGVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 642 bp, ARTIFICIAL SEQUENCE
  MOUSE HUMAN CHIMERA SEQUENCE
gacatccagatgacccagtctccatcctccttatctgcctctctgggagaaagagtcagtctcacttgtcgggcaagtcaggaaattagtgattacttaacctggcttcagcagaaaccagatggaactattaaacgcctgatctacgtcgcatccagtttagattctggtgtcccaaaaaggttcagtggcagtaggtctgggtcagattattctctcaccatcagcagccttgagtctgaagattttgcagactattactgtctacaatatgctaattatccgtggacgttcggtggaggcaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 214 aa, ARTIFICIAL SEQUENCE
  MOUSE HUMAN CHIMERA SEQUENCE
DIQMTQSPSSLSASLGERVSLTCRASQEISDYLTWLQQKPDGTIKRLIYVASSLDSGVPKRFSGSRSGSDYSLTISSLESEDFADYYCLQYANYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 10 aa, MOUSE SP.
GYIFTIAGIQ

SEQ ID NO: 10 - protein, 17 aa, MOUSE SP.
WINTHSGVPEYAEEFKG

SEQ ID NO: 11 - protein, 12 aa, MOUSE SP.
IYYGNNGGVMDY

SEQ ID NO: 12 - protein, 11 aa, MOUSE SP.
RASQEISDYLT

SEQ ID NO: 13 - protein, 7 aa, MOUSE SP.
VASSLDS

SEQ ID NO: 14 - protein, 9 aa, MOUSE SP.
LQYANYPWT

SEQ ID NO: 15 - protein, 121 aa, ARTIFICIAL SEQUENCE
  MOUSE HUMAN CHIMERA SEQUENCE
EVQLVESGGGLVQPGGSLRLSCAASGYIFTIAGIQWVRQAPGKGLEWVAWINTHSGVPEYAEEFKGRFTISRDNAKNSLYLQMNSLRAEDTAVYYCARIYYGNNGGVMDYWGQGTLVTVSS

SEQ ID NO: 16 - protein, 107 aa, ARTIFICIAL SEQUENCE
  MOUSE HUMAN CHIMERA SEQUENCE
DIQMTQSPSSLSASVGDRVTITCRASQEISDYLTWYQQKPGKAPKLLIYVASSLDSGVPSRFSGSGSGTDFTLTISSLQPEDFATYYCLQYANYPWTFGGGTKVEIK

SEQ ID NO: 17 - DNA, 363 bp, ARTIFICIAL SEQUENCE
  MOUSE HUMAN CHIMERA
gaggtgcagctggtggagtctgggggaggcttggtccagcctggggggtccctgagactctcctgtgcagcctctgggtatatcttcacaattgcgggaatacagtgggtccgccaggctccagggaaggggctggagtgggtggcctggataaacacccattctggagtgccagaatatgcagaagagttcaagggacgattcaccatctccagagacaacgccaagaactcactgtatctgcaaatgaacagcctgagagccgaggacacggctgtgtattactgtgcgagaatctactatggtaacaacgggggtgttatggactattggggccaaggaaccctggtcaccgtctcctca

SEQ ID NO: 18 - protein, 5 aa, ARTIFICIAL SEQUENCE
  CHEMICALLY SYNTHESIZED PEPTIDE
GGGGS

SEQ ID NO: 19 - protein, 121 aa, ARTIFICIAL SEQUENCE
  HUMAN MOUSE CHIMERA SEQUENCE
EVQLVESGGGLVQPGGSLRLSCAASGYIFTIAGIQWVRQAPGKGLEWVGWINTHSGVPEYAEEFKGRFTISLDNAKNSLYLQMNSLRAEDTAVYYCARIYYGNNGGVMDYWGQGTLVTVSS

SEQ ID NO: 20 - DNA, 321 bp, ARTIFICIAL SEQUENCE
  MOUSE HUMAN CHIMERA SEQUENCE
gacatccagatgacccagtctccatcctccctgtctgcatctgtaggagacagagtcaccatcacttgccgggcaagtcaggaaattagtgattacttaacctggtatcagcagaaaccagggaaagcccctaagctcctgatctatgtcgcatccagtttagattctggggtcccatcaaggttcagtggcagtggatctgggacagatttcactctcaccatcagcagtctgcaacctgaagattttgcaacttactactgtctacaatatgctaattatccgtggacgttcggcggagggaccaaggtggagatcaaa

SEQ ID NO: 21 - protein, 121 aa, ARTIFICIAL SEQUENCE
  MOUSE HUMAN CHIMERA SEQUENCE
EVQLVESGGGLVQPGGSLRLSCAASGYIFTIAGIQWVRQAPGKGLEWVGWINTHSGVPEYAEEFKGRFTISLDNAKNSAYLQMNSLRAEDTAVYYCARIYYGNNGGVMDYWGQGTLVTVSS

SEQ ID NO: 22 - DNA, 363 bp, ARTIFICIAL SEQUENCE
  HUMAN MOUSE CHIMERA SEQUENCE
gaggtgcagctggtggagtctgggggaggcttggtccagcctggggggtccctgagactctcctgtgcagcctctgggtatatcttcacaattgcgggaatacagtgggtccgccaggctccagggaaggggctggagtgggtgggatggataaacacccattctggagtgccagaatatgcagaagagttcaagggacgattcaccatctccctggacaacgccaagaactcagcatatctgcaaatgaacagcctgagagccgaggacacggctgtgtattactgtgcgagaatctactatggtaacaacgggggtgttatggactattggggccaaggaaccctggtcaccgtctcctca

SEQ ID NO: 23 - protein, 121 aa, ARTIFICIAL SEQUENCE
  HUMAN MOUSE CHIMERA SEQUENCE
EVQLVESGGGLVQPGGSLRLSCAASGYIFTIAGIQWVRQAPGKGLEWVGWINTHSGVPEYAEEFKGRFTFSLDNAKNSAYLQMNSLRAEDTAVYYCARIYYGNNGGVMDYWGQGTLVTVSS

SEQ ID NO: 24 - DNA, 363 bp, ARTIFICIAL SEQUENCE
  HUMAN MOUSE CHIMERA SEQUENCE
gaggtgcagctggtggagtctgggggaggcttggtccagcctggggggtccctgagactctcctgtgcagcctctgggtatatcttcacaattgcgggaatacagtgggtccgccaggctccagggaaggggctggagtgggtgggatggataaacacccattctggagtgccagaatatgcagaagagttcaagggacgattcaccttctccctggacaacgccaagaactcagcatatctgcaaatgaacagcctgagagccgaggacacggctgtgtattactgtgcgagaatctactatggtaacaacgggggtgttatggactattggggccaaggaaccctggtcaccgtctcctca

SEQ ID NO: 25 - protein, 37 aa, ARTIFICIAL SEQUENCE
  HUMAN MOUSE CHIMERA SEQUENCE
SLRAEDTAVYYCARIYYGNNGGVMDYWGQGTLVTVSS

SEQ ID NO: 26 - protein, 239 aa, STAPHYLOCCOCCUS AUREUS - MRSA
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLYDKEK

SEQ ID NO: 27 - protein, 107 aa, ARTIFICIAL SEQUENCE
  HUMAN MOUSE CHIMERA SEQUENCE
DIQMTQSPSSLSASVGDRVTITCRASQEISDYLTWLQQKPGKAIKRLIYVASSLDSGVPSRFSGSRSGTDFTLTISSLQPEDFATYYCLQYANYPWTFGGGTKVEIK

SEQ ID NO: 28 - DNA, 321 bp, ARTIFICIAL SEQUENCE
  HUMAN MOUSE CHIMERA SEQUENCE
gacatccagatgacccagtctccatcctccctgtctgcatctgtaggagacagagtcaccatcacttgccgggcaagtcaggaaattagtgattacttaacctggctccagcagaaaccagggaaagccatcaagcgcctgatctatgtcgcatccagtttagattctggggtcccatcaaggttcagtggcagtcgttctgggacagatttcactctcaccatcagcagtctgcaacctgaagattttgcaacttactactgtctacaatatgctaattatccgtggacgttcggcggagggaccaaggtggagatcaaa

SEQ ID NO: 29 - protein, 107 aa, ARTIFICIAL SEQUENCE
  HUMAN MOUSE CHIMERA SEQUENCE
DIQMTQSPSSLSASVGDRVTITCRASQEISDYLTWLQQKPGKAIKRLIYVASSLDSGVPSRFSGSGSGTDFTLTISSLQPEDFATYYCLQYANYPWTFGGGTKVEIK

SEQ ID NO: 30 - protein, 239 aa, STAPHYLOCOCCUS AUREUS
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 31 - DNA, 363 bp, ARTIFICIAL SEQUENCE
  HUMAN MOUSE CHIMERA SEQUENCE
gaggtgcagctggtggagtctgggggaggcttggtccagcctggggggtccctgagactctcctgtgcagcctctgggtatatcttcacaattgcgggaatacagtgggtccgccaggctccagggaaggggctggagtgggtgggatggataaacacccattctggagtgccagaatatgcagaagagttcaagggacgattcaccatctccctggacaacgccaagaactcactgtatctgcaaatgaacagcctgagagccgaggacacggctgtgtattactgtgcgagaatctactatggtaacaacgggggtgttatggactattggggccaaggaaccctggtcaccgtctcctca

(19) United States
(12) Patent Application Publication
(10) Pub. No.: US 2016/0039914 A1
(43) Pub. Date: Feb. 11, 2016

(54) HUMANIZED ANTIBODIES SPECIFIC FOR STAPHYLOCOCCAL ENTEROTOXIN B

(71) Applicants: ALBERT EINSTEIN COLLEGE OF MEDICINE OF YESHIVA UNIVERSITY, Bronx, NY (US); PFIZER, INC., Pearl River, NY (US)

(72) Inventors: Jenny MacIntyre, New York, NY (US); Fergus R. Byrne, Brooklyn, NY (US); Oleg Kovalenko, Arlington, MA (US); Avanish Varshney, Bronx, NY (US); Xiaobo Wang, New York, NY (US); Bettina Fries, New Rochelle, NY (US)

(21) Appl. No.: 14/774,283

(22) PCT Filed: Mar. 13, 2014
(86) PCT No.: PCT/US2014/025861

§ 371 Date: Sep. 10, 2015

Related U.S. Application Data
(60) Provisional application No. 61/926,446, filed on Jan. 13, 2014.
(60) Provisional application No. 61/784,338, filed on Mar. 14, 2013.

(51) Int. Cl. C07K 16/12; A61K 39/40; A61K 38/14; C07K 16/46

(52) U.S. Cl. CPC C07K 16/1271; C07K 16/464; A61K 39/40; A61K 38/14; C07K 2317/24; C07K 2317/76; C07K 2317/565; C07K 2317/569; C07K 2317/92

(57) ABSTRACT

Humanized Antibodies to SEB, fragments thereof, and compositions comprising such are provided. Therapies for staphylococcal infections and diseases are also provided. The presently claimed invention was made by or on behalf of the below listed parties to a joint research agreement. The joint research agreement was in effect on or before the date the claimed invention was made and the claimed invention was made as a result of activities undertaken within the scope of the joint research agreement.

### CROSS-REFERENCE TO RELATED APPLICATIONS

This application claims benefit of U.S. Provisional Application No. 61/784,338, filed Mar. 14, 2013, and of U.S. Provisional Application No. 61/926,446, filed Jan. 13, 2014, the contents of each of which are hereby incorporated by reference.

### PARTIES TO A JOINT RESEARCH AGREEMENT

The presently claimed invention was made by or on behalf of the below listed parties to a joint research agreement. The joint research agreement was in effect on or before the date the claimed invention was made and the claimed invention was made as a result of activities undertaken within the scope of the joint research agreement. The parties to the joint research agreement are PFIZER INC. and ALBERT EINSTEIN COLLEGE OF MEDICINE OF YESHIVA UNIVERSITY.

### BACKGROUND OF THE INVENTION

The disclosures of all patents, patent application publications and publications referred to in this application, including those cited to by number in parentheses, are hereby incorporated by reference in their entirety into the subject application to more fully describe the art to which the subject invention pertains.

The Staphylococcal enterotoxins (SEs) comprise a family of distinct toxins (A-E) all of which are excreted by various strains of Staphylococcus aureus (S. aureus) (1). Staphylococcal enterotoxin B (SEB) is a well characterized 28 kDa protein that is related to SEC1-3 on the basis of sequence homology (1, 2). SEB is a superantigen that triggers cytokine production and T-cell proliferation by cross-linking MHC class II molecules on antigen presenting cells and T-cell receptors (TCR) (2-5). In humans, SEB can trigger toxic shock, profound hypotension and multi-organ failure. SEB is the major enterotoxin associated with non-menstrual toxic shock syndrome and accounts for the majority of intoxications that are not caused by toxic shock syndrome toxin 1 (TSST-1). In addition, some reports indicate that SEB induces an IgE response and thereby might contribute to the pathogenesis of asthma, chronic rhinitis, and dermatitis (6-9). SEB is considered a select agent. The quantities needed to produce a desired effect are much lower than with synthetic chemicals. Also SEB can be easily produced in large quantities (10).

Currently there are no therapies available for treating enterotoxin-induced shock, but clinical data suggests that immunoglobulins can alleviate disease (11).

The present invention addresses this need for new therapies for treating SEB-induced diseases and pathologies, and provides humanized antibodies therefor.

### SUMMARY OF THE INVENTION

An isolated antibody is provided that specifically binds to a staphylococcal enterotoxin B (SEB) and comprises (A) (i) a heavy chain variable region (VH) comprising a VH complementarity determining region (CDR) one (CDR1) comprising SEQ ID NO:9, a VH CDR2 comprising SEQ ID NO:10, and VH CDR3 comprising SEQ ID NO:11; or (ii) a light chain variable region (VL) comprising a VL CDR1 comprising SEQ ID NO:12, a VL CDR2 comprising SEQ ID NO:13, and a VL CDR3 comprising SEQ ID NO:14, or (iii) a heavy chain variable region (VH) comprising a VH CDR1 comprising SEQ ID NO:9, a VH CDR2 comprising SEQ ID NO:10, a VH CDR3 comprising SEQ ID NO:11, a VL CDR1 comprising SEQ ID NO:12, a VL CDR2 comprising SEQ ID NO:13, and a VL CDR3 comprising SEQ ID NO:14,

and (B) an Fc portion having a sequence at least 90% identical to a human Fc region and/or a variable domain framework sequence having a sequence at least 90% identical to a human variable domain framework sequence, or an SEB-binding fragment of such antibody.

Also provided is a single-chain variable fragment (scFv) that (a) specifically binds to a staphylococcal enterotoxin B (SEB) and comprises a heavy chain variable region (VH) and a light chain variable region (VL) linked to each other by a linker peptide, wherein (i) the VH comprises a VH CDR1 comprising SEQ ID NO:9, a VH CDR2 comprising SEQ ID NO:10, a VH CDR3 comprising SEQ ID NO:11, or (ii) the VL comprises a VL CDR1 comprising SEQ ID NO:12, a VL CDR2 comprising SEQ ID NO:13, and a VL CDR3 comprising SEQ ID NO:14; or (iii) the VH comprises a VH CDR1 comprising SEQ ID NO:9, a VH CDR2 comprising SEQ ID NO:10, a VH CDR3 comprising SEQ ID NO:11, and the VL comprises a VL CDR1 comprising SEQ ID NO:12, a VL CDR2 comprising SEQ ID NO:13, and a VL CDR3 comprising SEQ ID NO:14, and (b) comprises a variable domain framework sequence thereof has a sequence at least 90% identical to a human variable domain framework sequence.

Also provided is a humanized isolated antibody that specifically binds to staphylococcal enterotoxin B and binds to an epitope that is the same as or overlaps with the epitope on staphylococcal enterotoxin B recognized by monoclonal antibody 20B1 v1.4/1.1 or monoclonal antibody 20B1 v1.6/1.1.

Also provided is an isolated antibody or antigen-binding portion thereof, comprising a sequence encoded by (i) plasmid hu20B1 VL v1.1 deposited on Mar. 13, 2013 with the ATCC and having ATCC Accession No. PTA-13616; (ii) plasmid hu20B1 VH v1.4 deposited on Mar. 13, 2013 with the ATCC and having ATCC Accession No. PTA-13617; or (iii) plasmid hu20B1 VH v1.6 deposited on Mar. 13, 2013 with the ATCC and having ATCC Accession No. PTA-13618.

Also provided is a pharmaceutical composition comprising the antibody or fragment or scFv as described herein.

Also provided is a cell line that recombinantly produces the antibody or fragment or scFv as described herein.

Also provided is a nucleic acid encoding the antibody or scFv as described herein.

Also provided is a method of treating a subject having, or at risk for, a staphylococcal disease, the method comprising administering to an subject having or at risk for a staphylococcal disease an amount of the antibody or fragment as described herein effective to treat a subject having, or at risk for, a staphylococcal disease.

Also provided is a method of treating a subject having, or at risk for, a staphylococcal disease, the method comprising administering to an subject having or at risk for a staphylococcal disease an amount of the antibody or fragment as described herein effective to treat a subject having, or at risk for, a staphylococcal disease, and further administering an effective amount of vancomycin.

Also provided is a method of treating a subject having, or at risk for, a staphylococcal disease, the method comprising administering to an subject having or at risk for a staphylococcal disease an amount of the scFv as described herein effective to treat a subject having, or at risk for, a staphylococcal disease.

Also provided is an isolated antibody that specifically binds to a staphylococcal enterotoxin B (SEB), or an isolated SEB-binding fragment of an antibody that that specifically binds to SEB and which comprises: a heavy chain variable region (VH) comprising a VH complementarity determining region one (CDR1), VH complementarity determining region two (CDR2), and complementarity determining region three (VH CDR3) of the VH sequence of SEQ ID NO: 15, 19, 21, 23, or 25; and a light chain variable region (VL) comprising a VL CDR1, VL CDR2, and VL CDR3.

A plasmid vector DNA is provided comprising an insert encoding hu20B1 VL v1.1 (ATCC accession number PTA-PTA-13616); a plasmid vector DNA comprising an insert encoding the hu20B1 VH v1.4 (ATCC accession number PTA-PTA-13617); or a plasmid vector DNA comprising an insert encoding the hu20B1 VH v1.6 having (ATCC accession number PTA-PTA-13618).

An isolated nucleic acid is provided encoding a VH or a VL of an antibody or fragment or scFv as described herein.

Also provided is a composition comprising any of the antibodies and/or antigen-binding fragments or scFv described herein.

Also provided is a pharmaceutical composition comprising any of the antibody or antigen-binding fragments described herein or scFv as described herein, and a pharmaceutically acceptable carrier.

Also provided is pharmaceutical composition comprising any of the antibody or antigen-binding fragments described herein or scFv as described herein, and an amount of vancomycin.

### BRIEF DESCRIPTION OF THE DRAWINGS

FIG. 1A-1B. Sequence alignments of a murine 20B1 and its humanized variants with human acceptor frameworks DP-54 for VH (panel A) and DPK9 for VL (panel B). Murine sequences are in italic, with differences from the human frameworks underlined. CDR sequences are in bold italic. Back-mutations in humanized 20B1 are boxed and their positions indicated by numbers at the top of the alignments.

FIG. 2. Direct SEB ELISA with chimeric and humanized 20B1 variants. muVH and muVL refer to murine V-domains in the context of chimeric heavy and light chains.

FIG. 3. Competition ELISA with murine, chimeric and humanized 20B1 antibody variants.

FIG. 4. Octet binding curves for murine, chimeric and humanized 20B1 antibodies at different concentrations of SEB.

FIG. 5: Effect of Mouse anti-SEB Mab Clone 20B1 and anti-E. tenella on SEB-induced Murine Cell Proliferation. Murine splenocyte single cell suspension was produced by macerating freshly excised spleen from normal female BALB/c mice that are 6-8 weeks from Albert Einstein College of Medicine (AECOM). RBCs were then lysed, solution strained, and then cells seeded into a 96-well plate. Test antibodies were then added to wells to the indicated concentration. Nanomolar concentration is based on assumed Molecular Weight of ≈145 kD. SEB was then added to final concentration of 25 ng/mL. Cells were incubated at 37° C., with 5% CO₂ for 4 days. The circles represent an average of triplicates of the relative fluorescence units (RFU). The triangles represent an average RFU of a monoclonal antibody directed against E. tenella, a physiologically irrelevant antibody. The dotted line represents the cellular IC₅₀. Percentage of control for zero percent is zero stimulant added. Percentage of control for 100 percent is 25 ng/mL SEB. After 4 days, readout was determined by a commercially available Vialight Cell kit (Lonza) and was used per the manufacturer's instructions. Cells were lysed and cellular ATP was detected as a surrogate for cell proliferation. Samples were read on the Wallac Envision luminometer. Curves were generated by XLfit with 4 parameter curve. Each experiment was done twice.

FIG. 6: Effect of chimeric 20B1 TM Fc, Containing Human Complementarity Determining Regions and Murine Variable Regions and anti-TT huIgG TM Fc on SEB-induced Murine Cell Proliferation. Murine splenocyte single cell suspension was produced by macerating freshly excised spleen from normal female BALB/c mice that are 6-8 weeks from AECOM. RBCs were then lysed, solution strained, and then cells seeded into a 96-well plate. Test antibodies were then added to wells to the indicated concentration. Nanomolar concentration is based on assumed Molecular Weight of ≈145 kD. SEB was then added to final concentration of 25 ng/mL. Cells were incubated at 37° C., with 5% CO₂ for 4 days. The circles represent an average of triplicates of the relative fluorescence units (RFU). The triangles represent an average RFU of a monoclonal antibody directed against E. tenella, a physiologically irrelevant antibody. The red dotted line represents the cellular IC₅₀. Percentage of control for zero percent is zero stimulant added. Percentage of control for 100 percent is 25 ng/mL SEB. After 4 days, readout was determined by a commercially available Vialight Cell kit (Lonza) and was used per the manufacturer's instructions. Cells were lysed and cellular ATP was detected as a surrogate for cell proliferation. Samples were read on the Wallac Envision luminometer. Curves were generated by XLfit with 4 parameter curve. Each experiment was done twice. These data clearly show this antibody almost fully inhibits SEB-induced murine splenocyte proliferation down to a level of 10%, with IC₅₀ value of 1.37 nM, which is within normal expected background signal for this assay system, relative to an isotype control antibody.

FIG. 7: Aggregated Data for Effect of Humanized 20B1 variant 1.4/1.1 on SEB-induced Murine Splenocytes Proliferation. Murine splenocyte single cell suspension was produced by macerating freshly excised spleen from normal female BALB/c mice that are 6-8 weeks from AECOM. RBCs were then lysed, solution strained, and then cells seeded into a 96-well plate. Test antibodies were then added to wells to the indicated concentration. Nanomolar concentration is based on assumed Molecular Weight of ≈145 kD. SEB was then added to final concentration of 25 ng/mL. Cells were incubated at 37° C., with 5% CO₂ for 4 days. Each graph represents an assay result. On each graph, the circles represent an average of triplicates of the relative fluorescence units (RFU). The dotted line represents the cellular IC₅₀. Percentage of control for zero percent is zero stimulant added. Percentage of control for 100 percent is 25 ng/mL SEB. After 4 days, readout was determined by a commercially available Vialight Cell kit (Lonza) and was used per the manufacturer's instructions. Cells were lysed and cellular ATP was detected as a surrogate for cell proliferation. Samples were read on the Wallac Envision luminometer. Curves were generated by XLfit with 4 parameter curve. This experiment was done four times to define the range of IC₅₀ and percent inhibition and the data from each experiment are shown in the four panels. These data demonstrate humanized 20B1 variant 1.4/1.1 antibody (hu20B1v1.4/1.1) fully inhibits SEB-induced murine splenocyte proliferation, with an IC₅₀ value of 0.7-1.5 nM.

FIG. 8: Aggregated data for humanized 20B1 antibody with 1.6/1.1 mutation (hu20B1v1.6/1.1). See FIG. 7 description for methods. Similarly to FIG. 7, this experiment was performed four times to define the range of IC₅₀ and percent inhibition and the data of each experiment are shown in the four panels.

FIG. 9A-9B: Effect of hu20B1v1.4/1.1 (FIG. 9A) and hu20B1v1.6/1.1 (FIG. 9B) on SEB-induced in vitro T cell proliferation in human PBMCs.

FIG. 10A-10B: In vivo toxin model (A) Survival (5 mice per group) (B) IFN-gamma inhibition from blood after 2 hr and 8 hr post infection.

FIG. 11A-11B: In vivo thigh model (A) WBC count and (B) cytokine IL-1ra level in the abscess.

FIG. 12A-12D: In vivo thigh infection model (n=10 per group) (A) CFU count was lower in abscesses of mAb-20B1, Hu-mAb 1.4/1.1 and 1.6/1.1 treated mice compared to treatment with PBS or IC (isotype control antibody); however, the difference was not significant. (20B1: mAb-20B1; IC: isotype control; VM: vancomycin; SI: Sham Incision). (B) Hu-mAb treatment significantly lowered the level of cytokine IL-1ra in the abscess of mice compared to PBS treated mice. Each point represents the analysis of an individual mouse. Statistically significant differences were calculated using the 2-tailed heteroschedastic t test. (C) The presence of SEB was significantly lower in mAb treated mice compared to PBS treated mice when detection mAB 6D3-IgG2a was used. (D) Unbound SEB was significantly higher only in the abscesses of vancomycin or PBS group compared to either mAb20B1 or Hu-mAb group (p<0.01). (20B1: mAb20B1; IC: isotype control; VM: vancomycin; SI: Sham Incision).

FIG. 13A-13B: Hu 1.6/1.1 in a S. aureus sepsis model (A) Survival (B) Survival benefit of Hu-1.6/1.1 and vancomycin combination: Mice (10 mice per group) treated with Hu-mAb 1.6/1.1 (i.v.) survived significantly longer when compared to isotype control mAb (IC) treated group and infected with S. aureus (MRSA strain 38, i.v. 5×10⁷ CFU) sepsis in dose dependent manner. FIG. 14B: Survival benefit was significantly enhanced in adjunctive combination therapy using low dose of Hu-mAb 1.6/1.1 (250 μg) and vancomycin (VM) when compared to PBS treated mice (p=0.002). Analysis of survival data were performed using Log-rank (Mantel-Cox Test).

### DETAILED DESCRIPTION OF THE INVENTION

Abbreviations used herein:
CFU—Colony Forming Unit
PBMC—Peripheral Blood Mononuclear Cell
SE—Staphylococcal enterotoxin;
SEB—Staphylococcal enterotoxin B;
TSST-1—toxic shock syndrome toxin;
SEBILS—SEB-induced lethal shock;
Ab—antibody;
mAb—monoclonal antibody;
FcγR—Fc gamma receptor.

An isolated antibody is provided that specifically binds to a staphylococcal enterotoxin B (SEB) and comprises (A) (i) a heavy chain variable region (VH) comprising a VH complementarity determining region (CDR) one (CDR1) comprising SEQ ID NO:9, a VH CDR2 comprising SEQ ID NO:10, and VH CDR3 comprising SEQ ID NO:11; or (ii) a light chain variable region (VL) comprising a VL CDR1 comprising SEQ ID NO:12, a VL CDR2 comprising SEQ ID NO:13, and a VL CDR3 comprising SEQ ID NO:14, or (iii) a heavy chain variable region (VH) comprising a VH CDR1 comprising SEQ ID NO:9, a VH CDR2 comprising SEQ ID NO:10, a VH CDR3 comprising SEQ ID NO:11, a VL CDR1 comprising SEQ ID NO:12, a VL CDR2 comprising SEQ ID NO:13, and a VL CDR3 comprising SEQ ID NO:14,

and (B) an Fc portion having a sequence at least 90% identical to a human Fc region and/or a variable domain framework sequence having a sequence at least 90% identical to a human variable domain framework sequence, or an SEB-binding fragment of such antibody. In an embodiment, the isolated antibody, or an SEB-binding fragment, comprises a variable domain framework sequence having a sequence identical to a human variable domain framework sequence.

In an embodiment, the isolated antibody or SEB-binding antibody fragment comprises a variable domain framework sequence having a sequence at least 90% identical to a human variable domain FR1, FR2, FR3 or FR4.

In an embodiment, the isolated antibody or SEB-binding antibody fragment comprises the VH sequence of SEQ ID NO:15, 19, 21, 23, or 25 or having a sequence at least 90% identical thereto.

In an embodiment, the isolated antibody or SEB-binding antibody fragment comprises the VL sequence of SEQ ID NO:16, 27, or 29 or has a sequence at least 90% identical thereto.

In an embodiment, the isolated antibody or SEB-binding antibody fragment comprises a VH comprising a VH CDR1 comprising SEQ ID NO:9, a VH CDR2 comprising SEQ ID NO:10, and a VH CDR3 comprising SEQ ID NO:11, and wherein the VL comprises a VL CDR1 comprising SEQ ID NO:12, a VL CDR2 comprising SEQ ID NO:13, and a VL CDR3 comprising SEQ ID NO:14.

In an embodiment, the variable domain framework of the VH comprises (a) the amino acid sequence of SEQ ID NO: 15, 19, 21, 23, or 25 or (b) a variant of one of SEQ ID NOS: 15, 19, 21, 23, or 25 with one to five conservative amino acid substitutions in residues that are not within a CDR1, CDR2 or CDR3 thereof. In an embodiment, the one to five conservative amino acid substitutions in the variable domain framework of the VH that are not within a CDR1, CDR2 or CDR3 include one or more of the following: A49G, I69F, R71L, and L78L. (See SEQ ID NOS:19, 21, 23 and 25, for example).

In an embodiment, the variable domain framework of the VL comprises (a) the amino acid sequence of SEQ ID NO: 16, 27 or 29, or (b) a variant of one of SEQ ID NOS: 16, 27 or 29, with one to five conservative amino acid substitutions in residues that are not within a CDR1, CDR2 or CDR3 thereof. In an embodiment, the one to five conservative amino acid substitutions in the variable domain framework of the VL that are not within a CDR1, CDR2 or CDR3 include one or more of the following: Y36L, P44I, L46R, G66R. (See SEQ ID NOS:27 and 29, for example).

In an embodiment, the isolated antibody or SEB-binding antibody fragment comprises an Fc region having a sequence identical to a human Fc region.

In an embodiment, the Fc region of the antibody is glycosylated.

In an embodiment, the isolated antibody or SEB-binding antibody fragment comprises a variable domain framework sequence having a sequence identical to a human variable domain framework sequence FR1, FR2, FR3 or FR4.

In an embodiment, the isolated antibody or SEB-binding antibody fragment has an isotype that is selected from the group consisting of IgG₂, IgG_{2Δa}, IgG₄, IgG_{4Δb}, IgG_{4Δc}, IgG₄ S228P, IgG_{4Δb} S228P and IgG_{4Δc} S228P.

In an embodiment, the isolated antibody or SEB-binding antibody fragment comprises is capable of blocking staphylococcal enterotoxin B from cross-linking a major histocompatibility complex class II molecule on an antigen presenting cell and a T-cell receptor on a second cell.

In an embodiment, the invention is the isolated antibody. In an embodiment, the invention is the SEB-binding antibody fragment.

Also provided is a single-chain variable fragment (scFv) that (a) specifically binds to a staphylococcal enterotoxin B (SEB) and comprises a heavy chain variable region (VH) and a light chain variable region (VL) linked to each other by a linker peptide, wherein (i) the VH comprises a VH CDR1 comprising SEQ ID NO:9, a VH CDR2 comprising SEQ ID NO:10, a VH CDR3 comprising SEQ ID NO:11, or (ii) the VL comprises a VL CDR1 comprising SEQ ID NO:12, a VL CDR2 comprising SEQ ID NO:13, and a VL CDR3 comprising SEQ ID NO:14; or (iii) the VH comprises a VH CDR1 comprising SEQ ID NO:9, a VH CDR2 comprising SEQ ID NO:10, a VH CDR3 comprising SEQ ID NO:11, and the VL comprises a VL CDR1 comprising SEQ ID NO:12, a VL CDR2 comprising SEQ ID NO:13, and a VL CDR3 comprising SEQ ID NO:14, and (b) comprises a variable domain framework sequence thereof has a sequence at least 90% identical to a human variable domain framework sequence.

In an embodiment, the scFv comprises a variable domain framework sequence having a sequence identical to a human variable domain FR1, FR2, FR3 or FR4.

In an embodiment, the scFv comprises a linker peptide from 5 to 30 amino acid residues long.

In an embodiment, the scFv comprises a linker peptide comprising one or more of glycine, serine and threonine residues.

Also provided is a humanized isolated antibody that specifically binds to staphylococcal enterotoxin B and binds to an epitope that is the same as or overlaps with the epitope on staphylococcal enterotoxin B recognized by monoclonal antibody 20B1 v1.4/1.1 or monoclonal antibody 20B1 v1.6/1.1.

In an embodiment, the antibody of the invention recognizes an epitope comprising amino acid residues 135, 137, 186, 235 and 236 of the staphylococcal enterotoxin B amino acid sequence shown in SEQ ID NO: 30. In an embodiment, the antibody recognizes an epitope comprising amino acid residues 135, 137, 186, 235 and 236 of the staphylococcal enterotoxin B amino acid sequence shown in SEQ ID NO: 26.

In an embodiment, the antibody binds to staphylococcal enterotoxin B with an equilibrium dissociation constant of about 250 nM or less. In an embodiment, the antibody binds to staphylococcal enterotoxin B with an equilibrium dissociation constant of about 900 pM or less. In an embodiment, the antibody binds to staphylococcal enterotoxin B with an equilibrium dissociation constant of about 450 pM or less.

Also provided is an isolated chimeric antibody as described herein.

Also provided is an isolated antibody or antigen-binding portion thereof, comprising a sequence encoded by (i) plasmid hu20B1 VL v1.1 deposited on Mar. 13, 2013 with the ATCC and having ATCC Accession No. PTA-13616; (ii) plasmid hu20B1 VH v1.4 deposited on Mar. 13, 2013 with the ATCC and having ATCC Accession No. PTA-13617; or (iii) plasmid hu20B1 VH v1.6 deposited on Mar. 13, 2013 with the ATCC and having ATCC Accession No. PTA-13618.

In an embodiment of the antibodies, or antibody fragments described herein, the antibody does not bind staphylococcal enterotoxin A (SEA) or toxic shock syndrome toxin 1 (TSST-1).

Also provided is a pharmaceutical composition the antibody or fragment or scFv as described herein.

Also provided is a pharmaceutical composition comprising any of the antibody or antigen-binding fragments described herein or scFv as described herein, and a pharmaceutically acceptable carrier

Also provided is pharmaceutical composition comprising any of the antibody or antigen-binding fragments described herein or scFv as described herein, and an amount of vancomycin. In an embodiment of the pharmaceutical composition, the combined amount of antibody, or antigen-binding fragment thereof, or scFv, and Vancomycin is a therapeutically effective synergistic amount.

Also provided is a cell line that recombinantly produces the antibody or fragment or scFv as described herein.

Also provided is a nucleic acid encoding the antibody or scFv as described herein.

Also provided is a method of treating a subject having, or at risk for, a staphylococcal disease, the method comprising administering to an subject having or at risk for a staphylococcal disease an amount of the antibody or fragment as described herein effective to treat a subject having, or at risk for, a staphylococcal disease.

Also provided is a method of treating a subject having, or at risk for, a staphylococcal disease, the method comprising administering to an subject having or at risk for a staphylococcal disease an amount of the scFv as described herein effective to treat a subject having, or at risk for, a staphylococcal disease.

In an embodiment, the subject is suffering from a staphylococcal disease. In an embodiment, the subject is suffering from a staphylococcal infection. In an embodiment, the subject is a mammal. In an embodiment, the mammal is selected from the group consisting of a human, a cow, a dog, a pig, and a cat. In an embodiment, the staphylococcal disease is selected from the group consisting of staphylococcal abscess, staphylococcal bacteremia, staphylococcal sepsis, staphylococcal eye infection, staphylococcal skin infection, staphylococcal soft tissue infection, staphylococcal osteomyelitis, staphylococcal mastitis, staphylococcal heart infection, and staphylococcal lung disease. In an embodiment, the staphylococcal sepsis is staphylococcal enterotoxin B induced lethal shock (SEBILS). In an embodiment, the staphylococcal disease is asthma, endocarditis, chronic rhinitis, or dermatitis. In an embodiment, the method further comprises administering an effective amount of an agent which is an anti-infective agent, an antibiotic agent, or an antimicrobial agent. In an embodiment, the agent is selected from the group consisting of trimethoprim-sulfamethoxazole, linezolid, vancomycin, telavancin, cefazolin, nafcillin, methicillin, dicloxacillin, doxycycline, minocycline, tigecycline, lysostaphin, clindamycin, daptomycin, quinupristin/dalfopristin, and penicillin.

In an embodiment, the antibody of the invention is administered in combination with Vancomycin. The combination of the antibody and Vancomycin provides a surprising synergistic effect greater than the additive effect of each compound alone. In addition, a decreased amount of the antibody can be used when administered in combination with Vancomycin when compared to administration of the antibody in the absence of Vancomycin therapy.

“Combination therapy” embraces the administration of an anti-SEB antibody, preferably, an antibody of the invention, such as, but not limited to, hu20B1 1.4/1.1 and 1.6/1.1, and another therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention.

“Combination therapy” embraces administration of these therapeutic agents in a sequential manner, that is, wherein each therapeutic agent is administered at a different time, as well as administration of these therapeutic agents, or at least two of the therapeutic agents, in a substantially simultaneous manner. Sequential or substantially simultaneous administration of each therapeutic agent can be effected by any appropriate route including, but not limited to, oral routes, intravenous routes, intramuscular, subcutaneous routes, and direct absorption through mucous membrane tissues. The therapeutic agents can be administered by the same route or by different routes. For example, a first therapeutic agent (e.g., an antibody of the invention) can be administered intravenously, and a second agent (e.g., vancomycin) can be administered intravenously or orally. Further, a first therapeutic agent of the combination selected (e.g., an antibody of the invention) may be administered by intravenous injection while the other therapeutic agents of the combination (e.g., an anti-infective agent, an antibiotic agent, or an antimicrobial agent) may be administered orally. Alternatively, for example, both the therapeutic agents may be administered by intravenous or subcutaneous injection.

In the present specification the term “sequential” means, unless otherwise specified, characterized by a regular sequence or order, e.g., if a dosage regimen includes the administration of an antibody of the invention and an antibiotic (e.g., Vancomycin), a sequential dosage regimen could include administration of the antibody before, simultaneously, substantially simultaneously, or after administration of the antibiotic agent, but both agents will be administered in a regular sequence or order. The term “separate” means, unless otherwise specified, to keep apart one from the other. The term “simultaneously” means, unless otherwise specified, happening or done at the same time, i.e., the compounds of the invention are administered at the same time. The term “substantially simultaneously” means that the compounds are administered within minutes of each other (e.g., within 60 minutes of each other, within 45 minutes of each other, within 30 minutes of each other, within 20 minutes of each other, within 15 minutes of each other, within 10 minutes of each other, and within 5 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the antibody and the antibiotic (e.g., vacomycin) agent.

“Combination therapy” also can embrace the administration of the therapeutic agents as described above in further combination with other biologically active ingredients (such as, but not limited to, a second and different antibody, antibiotic, anti-infective, etc.) and non-drug therapies (such as, but not limited to, surgery).

Also provided is an isolated antibody that specifically binds to a staphylococcal enterotoxin B (SEB), or an isolated SEB-binding fragment of an antibody that that specifically binds to SEB and which comprises: a heavy chain variable region (VH) comprising a VH complementarity determining region one (CDR1), VH complementarity determining region two (CDR2), and complementarity determining region three (VH CDR3) of the VH sequence of SEQ ID NO: 15, 19, 21, 23, or 25; and a light chain variable region (VL) comprising a VL CDR1, VL CDR2, and VL CDR3. In an embodiment, the antibody is chimeric or is humanized.

A plasmid vector DNA is provided comprising an insert encoding hu20B1 VL v1.1 (ATCC accession number PTA-13616); a plasmid vector DNA comprising an insert encoding the hu20B1 VH v1.4 (ATCC accession number PTA-13617); or a plasmid vector DNA comprising an insert encoding the hu20B1 VH v1.6 having (ATCC accession number PTA-13618).

An isolated nucleic acid is provided encoding a VH or a VL of an antibody or fragment or scFv or diabody as described herein.

An isolated nucleic acid is provided encoding a VH and a VL of an antibody or fragment or scFv or diabody as described herein.

Also provided is a method of treating a disease associated with a staphylococcus infection in a subject having the disease, or preventing a disease associated with a staphylococcus infection in a subject at risk of the disease, comprising administering to the subject an amount of an antibody directed against SEB as described herein or antigen-binding fragment thereof as described herein, effective to treat the disease. In an embodiment, the antibody is a monoclonal antibody.

In an embodiment, the invention includes a method of treating a disease associated with a staphylococcus infection in a subject having the disease, or preventing a disease associated with a staphylococcus infection in a subject at risk of the disease, comprising administering to the subject an amount of an antibody directed against SEB as described herein or antigen-binding fragment thereof as described herein, effective to treat the disease and a therapeutically effective amount of Vancomycin. In an embodiment, the antibody is a monoclonal antibody.

In an embodiment, the disease is sepsis, SEB-mediated shock, a staphylococcus aureus infection, staphylococcus aureus bacteremia, or staphylococcus aureus-associated atopic dermatitis. In an embodiment, the disease is staphylococcus aureus infection. In an embodiment, the disease is staphylococcus aureus skin infection. In an embodiment, the staphylococcus aureus is methicillin-resistant staphylococcus aureus. In an embodiment, the staphylococcus aureus is methicillin-sensitive staphylococcus aureus.

As used herein, “at least 90% identical to” encompasses a sequence that has at least 90%, 91%, 92%, 93%, 94%, 95%, 96%, 97%, 98% or 99% identity with, or is 100% identical to, the referenced sequence. Accordingly, the individual embodiments of at least 90% identical to, at least 91% identical to, at least 92% identical to, at least 93% identical to, at least 94% identical to, at least 95% identical to, at least 96% identical to, at least 97% identical to, at least 98% identical to, at least 99% identical to, and 100% identical to, are each all encompassed by the invention.

The antigen, in regard to the term “antigen-binding fragment” as used herein, is SEB or a portion thereof.

The terms “Staphylococcal enterotoxin B antibody” and “anti-Staphylococcal enterotoxin B antibody” are used interchangeably herein.

In an embodiment of the antibodies, fragments, methods and compositions described herein, the SEB comprises the sequence set forth in SEQ ID NO:30 or fragments thereof. In an embodiment of the antibodies, fragments, methods and compositions described herein, the SEB comprises the sequence set forth in SEQ ID NO:26 or fragments thereof.

In an embodiment of the antibodies, fragments, methods and compositions described herein, the fragment of the antibody comprises an Fab, an Fab′, an F(ab′)₂, an F_{d}, an Fᵥ, or a complementarity determining region (CDR). In an embodiment, the fragment comprises a CDR3 of a V_{H} chain. In an embodiment the fragment further comprises one of, more than one of, or all of CDR1, CDR2 of V_{H} and CDR1, CDR2 and CDR3 of a V_{L}. As used herein, an F_{d} fragment means an antibody fragment that consists of the V_{H} and C_{H1} domains; an Fᵥ fragment consists of the V_{L} and V_{H} domains of a single arm of an antibody; and a dAb fragment (Ward et al., Nature 341:544-546 (1989) hereby incorporated by reference in its entirety) consists of a V_{H} domain. In some embodiments, fragments are at least 5, 6, 8 or 10 amino acids long. In other embodiments, the fragments are at least 14, at least 20, at least 50, or at least 70, 80, 90, 100, 150 or 200 amino acids long.

In an embodiment of the methods, the antibody or antigen-binding fragment thereof is, or antibodies or antigen-binding fragments thereof are, administered prophylactically. In an embodiment, the antibody or antigen-binding fragment thereof is, or antibodies or antigen-binding fragments thereof are, administered after the disease has manifested. In an embodiment, the subject is administered an antibody or antibodies and the antibody or antibodies are chimeric monoclonal antibodies, humanized monoclonal antibodies or human monoclonal antibodies.

In an embodiment of the methods, the antibody, antibodies, antibody fragment or antibody fragments are administered as an adjuvant therapy to a primary therapy for the disease or condition.

As used herein, the term “isolated antibody” refers to an antibody that by virtue of its origin or source of derivation has one to four of the following characteristics: (1) is not associated with naturally associated components that accompany it in its native state, (2) is free of other proteins from the same species, (3) is expressed by a cell from a different species, or (4) does not occur in nature.

As used herein, the term “antibody” refers to an intact antibody, i.e., with complete Fc and Fv regions. “Fragment” refers to any portion of an antibody which is less than the whole antibody but which is an SEB-binding portion. In an embodiment, the fragment competes with the intact antibody, of which it is a fragment, for specific binding to SEB. As such a fragment can be prepared, in non-limiting examples, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced, in non-limiting examples, by recombinant DNA techniques or by enzymatic or chemical cleavage of intact antibodies or by molecular biology techniques.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine of the Fc region may be removed, for example, during production or purification of the antibody, or by recombinantly engineering the nucleic acid encoding a heavy chain of the antibody. Accordingly, an intact antibody as used herein may be an antibody with or without the otherwise C-terminal cysteine.

In an embodiment, the antibodies of the invention described herein comprise a human Fc region or a variant human Fc region. A variant human Fc region comprises an amino acid sequence which differs from that of a native sequence Fc region by virtue of at least one amino acid modification, yet retains at least one effector function of the native sequence human Fc region. Preferably, the variant Fc region has at least one amino acid substitution compared to a native sequence Fc region or to the Fc region of a parent polypeptide, e.g. from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

From N-terminus to C-terminus, both the mature light and heavy chain variable domains comprise the regions FR1, CDR1, FR2, CDR2, FR3, CDR3 and FR4. In an embodiment, the assignment of amino acids to each domain is in accordance with the definitions of Kabat, Sequences of Proteins of Immunological Interest (National Institutes of Health, Bethesda, Md. (1987 and 1991), hereby incorporated by reference in its entirety). In an embodiment, the assignment of amino acids to each domain is in accordance with the definitions of Chothia & Lesk, J. Mol. Biol. 196:901-917 (1987), or Chothia et al., Nature 342:878-883 (1989), each of which are hereby incorporated by reference in their entirety.

The framework regions of the antibodies of the invention having a sequence identical to a human framework region may include amino acid residues not encoded by human germline sequences (e.g., mutations introduced by random or site-specific mutagenesis). A sequence “identical to a human variable domain framework sequence” is a sequence having at least a FR1, a FR2, a FR3 or a FR4 sequence thereof identical to at least (i) a human FR1, (ii) a human FR2, (iii) a human FR3, or (iv) a human FR4, respectively, or comprising a sequences identical to two, three or four of (i) a human FR1, (ii) a human FR2, (iii) a human FR3, and (iv) a human FR4.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpy contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

In an embodiment, the antibodies as described herein are monoclonal antibodies. The term “monoclonal antibody” unless otherwise indicated by context is not intended to be limited as regards to the source of the antibody or the manner in which it is made (e.g., by hybridoma, phage selection, recombinant expression, transgenic animals, etc.). The term “monoclonal antibody” as used herein refers to an antibody member of a population of substantially homogeneous antibodies, i.e., the subject antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means once the sequence thereof is identified.

In an embodiment, the antibodies as described herein are recombinant antibodies. The term “recombinant antibody”, as used herein, includes antibodies that are prepared, expressed, created or isolated by recombinant means. “Humanized” forms antibodies as used herein, unless otherwise indicated, are chimeric antibodies that contain minimal sequence (CDRs) derived from non-human immunoglobulin. In one embodiment, a humanized antibody is a human immunoglobulin (recipient antibody) in which CDR residues from a hypervariable region (HVR) of the recipient are replaced by CDR residues from a a non-human species (donor antibody) such as mouse, rat, rabbit, or nonhuman primate having the desired specificity, affinity, and/or capacity. In some instances, FR residues of the human immunoglobulin variable domain are replaced by corresponding non-human residues, for example by back-mutation as described herein. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which references and patents are hereby incorporated by reference in their entirety. Other techniques to humanize a monoclonal antibody are described in U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety.

A single-chain antibody (scFv) is a variable domain light chain (V_{L}) and a variable domain heavy chain (V_{H}) which are linked N-C or C-N, respectively, via a peptide linker. In an embodiment the linker of the scFv is 5-30 amino acids in length. In an embodiment the linker of the scFv is 10-25 amino acids in length. In an embodiment the peptide linker comprises glycine, serine and/or threonine residues. For example, see Bird et al., Science, 242: 423-426 (1988) and Huston et al., Proc. Natl. Acad. Sci. USA, 85:5879-5883 (1988), each of which are hereby incorporated by reference in their entirety.

As used herein, the terms “specific for”, “specifically binds”, or “preferentially binds” refers to the property of an antibody or fragment of binding to the specified antigen (SEB) with a dissociation constant that is <1 μM, preferably <1 nM, more preferably <10 pM, and most preferably <1 pM. An antibody is “specific for” SEB or “specifically binds” SEB if it binds with greater affinity, avidity, more readily, and/or with greater duration than it binds to other substances. In an embodiment, the Kd of the antibody for SEB is 900 pM or less, 800 pM or less, 700 pM or less, 600 pM or less, 500 pM or less, 400 pM or less, 300 pM or less, 200 pM or less, or 100 pM or less. The binding affinity (KD) of an anti-SEB antibody of the invention to SEB can be about 0.001 to about 200 nM. In some embodiments, the binding affinity is any of about 200 nM, about 100 nM, about 50 nM, about 10 nM, about 1 nM, about 500 pM, about 100 pM, about 60 pM, about 50 pM, about 20 pM, about 15 pM, about 10 pM, about 5 pM, about 2 pM, or about 1 pM. In some embodiments, the binding affinity is less than any of about 250 nM, about 200 nM, about 100 nM, about 50 nM, about 10 nM, about 1 nM, about 500 pM, about 100 pM, about 50 pM, about 20 pM, about 10 pM, about 5 pM, or about 2 pM.

The term “K_{d}”, as used herein, is intended to refer to the dissociation constant of an antibody-antigen interaction. One way of determining the K_{d} or binding affinity of antibodies to SEB is by measuring binding affinity of monofunctional Fab fragments of the antibody. (The affinity constant is the inverted dissociation constant). To obtain monofunctional Fab fragments, an antibody (for example, IgG) can be cleaved with papain or expressed recombinantly. The affinity of an anti-SEB Fab fragment of an antibody can be determined by surface plasmon resonance (BIAcore3000™ surface plasmon resonance (SPR) system, BIAcore Inc., Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. SEB can be diluted into 10 mM sodium acetate pH 4.0 and injected over the activated chip at a concentration of 0.005 mg/mL. Using variable flow time across the subject chip channels, two ranges of antigen density can be achieved: 100-200 response units (RU) for detailed kinetic studies and 500-600 RU for screening assays. Serial dilutions (0.1-10× estimated Kₐ) of purified Fab samples are injected for 1 min at 100 microliters/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6. 99-110, the content of which is hereby incorporated in its entirety) using the BIA evaluation program. Equilibrium dissociation constant (Kₐ) values are calculated as k_{off}/kₒₙ. This protocol is suitable for use in determining binding affinity of an antibody or fragment to any SEB. Other protocols known in the art may also be used. For example, ELISA of SEB with mAb can be used to determine the K_{D} values. The K_{d} values reported herein used this ELISA-based protocol.

Preferably, the anti-SEB antibody or fragment thereof of the invention should exhibit any one or more of the following characteristics: (a) bind to Staphylococcal enterotoxin B; (b) block Staphylococcal enterotoxin B interaction with a cell surface receptor and downstream signaling events; (c) block Staphylococcal enterotoxin B mediated T lymphocyte stimulation; (d) block the Staphylococcal enterotoxin B mediated release of cytokines, e.g., interleukin 2, tumor necrosis factor β, and interferons; and (e) block Staphylococcal enterotoxin B mediated interaction between MHC II on antigen presenting cells and T-cell receptors on CD4 and CD8 T lymphocytes.

The antibody preferably reacts with Staphylococcal enterotoxin B in a manner that blocks Staphylococcal enterotoxin B interaction with a cell surface. In some embodiments, the anti-Staphylococcal enterotoxin B antibody specifically recognizes Staphylococcal enterotoxin B from a methicillin-resistant S. aureus strain (e.g., USA300) and Staphylococcal enterotoxin B from a methicillin sensitive S. aureus strain (e.g., PFESA0140). Preferably, the Staphylococcal enterotoxin B antibody binds one or more different Staphylococcal enterotoxin B variants. The Staphylococcal enterotoxin B antibodies of the invention exhibit one or more of the following characteristics: (a) bind to Staphylococcal enterotoxin B; (b) block Staphylococcal enterotoxin B interaction with a cell surface receptor and downstream signaling events; (c) block Staphylococcal enterotoxin B-mediated T lymphocyte stimulation; (d) block the Staphylococcal enterotoxin B-mediated release of cytokines, e.g., interleukin 2, tumor necrosis factor β, and interferons; and (e) block Staphylococcal enterotoxin B mediated interaction between MHC II on antigen presenting cells and T-cell receptors on CD4 and CD8 T lymphocytes. Preferably, Staphylococcal enterotoxin B antibodies have two or more of these features. More preferably, the antibodies have three or more of the features. More preferably, the antibodies have four or more of the features. More preferably, the antibodies have five or more of the features. More preferably, the antibodies have six or more of the features. Most preferably, the antibodies have all seven features.

The antibody or fragment of the invention can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000, hereby incorporated by reference in its entirety).

The invention encompasses compositions comprising the antibodies, fragments or scFv described herein. In an embodiment, the composition is a pharmaceutical composition. In an embodiment the composition or pharmaceutical composition comprising one or more of the antibodies or fragments described herein is substantially pure with regard to the antibody or fragment. A composition or pharmaceutical composition comprising one or more of the antibodies or fragments described herein is “substantially pure” with regard to the antibody or fragment when at least about 60 to 75% of a sample of the composition or pharmaceutical composition exhibits a single species of the antibody or fragment. A substantially pure composition or pharmaceutical composition comprising one or more of the antibodies or fragments described herein can comprise, in the portion thereof which is the antibody or fragment, 60%, 70%, 80% or 90% of the antibody or fragment of the single species, more usually about 95%, and preferably over 99%. Antibody purity or homogeneity may tested by a number of means well known in the art, such as polyacrylamide gel electrophoresis or HPLC.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation or aggregation over a period of time during storage and transportation prior to use. The compositions or pharmaceutical compositions can comprise one or more of any combination of salts, surfactants, pH and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for parenteral administration, including intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

In an embodiment the ScFvs or fragments of antibodies disclosed herein are lyophilized and/or freeze dried and are reconstituted for use.

The invention encompasses compositions comprising the antibodies, fragments or scFv described herein in a pharmaceutically acceptable carrier. As used herein, “pharmaceutically acceptable carrier” includes any material (including mixtures) which, when combined with an active ingredient, allows the ingredient to retain biological activity and is non-reactive with the subject's immune system. Examples include, but are not limited to, any of the standard pharmaceutical carriers such as one or more of phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate.

The antibodies, or fragments of antibodies, or compositions, or pharmaceutical compositions described herein can also be lyophilized or provided in any suitable forms including, but not limited to, injectable solutions or inhalable solutions, gel forms and tablet forms.

The practice of the present invention can employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

In one aspect, the invention provides a method for treating or preventing staphylococcal disease in a subject, the method comprising administering to the subject an effective amount of a Staphylococcal enterotoxin B antibody. “Staphylococcal enterotoxin B antibody” as used herein is an antibody or fragment of the invention as described herein. Staphylococcal disease refers to a disease, infection or condition caused by S. aureus. Examples of staphylococcal disease include, without limitation, staphylococcal gastroenteritis, staphylococcal bloodstream infections, including, e.g., bacteremia and septicemia; sepsis, staphylococcal lung disease, including, e.g., staphylococcal pneumonia; staphylococcal abscess, including, e.g., skin abscess, abdominal abscess, breast abscess, kidney abscess, heart abscess, liver abscess, lung abscess, brain abscess, and spleen abscess; staphylococcal skin infections, including, e.g., pimples, abscess, impetigo, boils, furuncles, cellulitis, atopic dermatitis, and dermonecrosis; staphylococcal eye infections, including, e.g., keratitis, blepharitis, conjunctivitis, and endophthalmitis; staphylococcal sepsis; septic arthritis, staphylococcal central nervous system (CNS) infections; mastitis; endocarditis; osteomyelitis; chorioamnionitis; neonatal sepsis; staphylococcal food poisoning; urinary tract infection; mastitis; TSS and meningitis. As used herein “sepsis” is the medically recognized condition characterized by a systemic inflammatory response to for example a pathogenic bacteria such as a staphylococcal pathogen.

In some embodiments, the staphylococcal disease is staphylococcal gastroenteritis. In other embodiments, the staphylococcal disease is staphylococcal sepsis, including, e.g. Staphylococcal enterotoxin B induced lethal shock (SEBILS). In other embodiments, the staphylococcal disease is staphylococcal abscess, including, e.g., skin abscess, abdominal abscess, breast abscess, kidney abscess, heart abscess, liver abscess, lung abscess, brain abscess, and spleen abscess. In other embodiments, the staphylococcal disease is staphylococcal bacteremia. In other embodiments, the staphylococcal disease is staphylococcal pneumonia. In other embodiments, the staphylococcal disease is staphylococcal osteomyelitis. In other embodiments, the staphylococcal disease is staphylococcal endocarditis. In other embodiments, the staphylococcal disease is staphylococcal keratitis, blepharitis, conjunctivitis, or endophthalmitis. In other embodiments, the staphylococcal disease is septic arthritis. In other embodiments, the staphylococcal disease is staphylococcal CNS infection.

In some embodiments, the subject involved in methods of the invention is considered to be at risk for a staphylococcal disease. “At risk” is an art-recognized term in the medical literature. Such subjects include, but are not limited to, an subject who is hospitalized or will be hospitalized, an subject who is or will be put in an intensive care unit, an subject who will undergo surgery, an subject who will be anesthetized or under general anesthesia, an subject over the age of 65, an subject with a compromised immune system, a pediatric subject, an subject who is or may be put on a respirator or other mechanical ventilator, an subject in whom an endotracheal tube will or has been placed, an subject who is or will be immobilized, an subject who will undergo, is undergoing, or has undergone chemotherapy or radiation/myeloablative therapy, an subject who will take, is taking, or has taken one or more immunosuppressants, particularly for a significant period of time (longer than a month), an subject with an infected wound, an subject with a minor injury, an subject who has an existing infection anywhere in the body, an subject who is or will be catheterized (e.g., with a urinary or indwelling IV catheter), an subject who will undergo a dental procedure, an subject who has an incised boil, an subject who has had his spleen removed, an subject taking steroids, an subject with diabetes, an subject with AIDS, and subject with cirrhosis, an subject with burns, and subject with severe injuries, and an subject with an infection such as, for example, pneumonia, meningitis, cellulitis, or urinary tract infection.

In some embodiments, methods may involve identifying a subject at risk for a staphylococcal disease. Additionally, methods may include evaluating a subject for risk factors for a staphylococcal disease, evaluating an subject for symptoms of a staphylococcal disease, or diagnosing the subject with a staphylococcal disease. In certain embodiments, methods may involve implementing steps in which the disease is staphylococcal pneumonia, staphylococcal abscess, bacteremia, sepsis, osteomyelitis, endocarditis, or keratitis.

In some embodiments, therapeutic administration of the Staphylococcal enterotoxin B antibody advantageously results in reduced incidence and/or amelioration of one or more symptoms of staphylococcal enterotoxin B induced lethal shock including, for example, fever, chills, rigors, fatigue, malaise, nausea, vomiting difficulty breathing, anxiety or altered mental status.

A subject suffering from or at risk for staphylococcal abscess can be treated with a Staphylococcal enterotoxin B antibody. An subject suitable for Staphylococcal enterotoxin B antibody therapy is selected using clinical criteria and prognostic indicators of Staphylococcal enterotoxin B induced lethal shock that are well known in the art. Assessment of staphylococcal abscess severity may be performed based on tests known in the art. In some embodiments, ameliorating, controlling, reducing incidence of, or delaying the development or progression of Staphylococcal enterotoxin B induced lethal shock and/or symptoms of Staphylococcal enterotoxin B induced lethal shock is measured by monitoring metabolic, cardiac and respiratory function.

In some embodiments, therapeutic administration of the anti-Staphylococcal enterotoxin B antibody advantageously results in reduced incidence and/or amelioration of one or more symptoms of staphylococcal bacteremia including, for example, fever, chills, malaise, abdominal pain, nausea, vomiting, diarrhea, anxiety, shortness of breath and confusion.

A subject suffering from or at risk for staphylococcal bacteremia can be treated with an anti-Staphylococcal enterotoxin B antibody. An subject suitable for Staphylococcal enterotoxin B antibody therapy is selected using clinical criteria and prognostic indicators of staphylococcal bacteremia that are well known in the art, including blood culture to check for presence of bacteria. Assessment of staphylococcal bacteremia severity may be performed based on tests known in the art.

In some embodiments, therapeutic administration of the Staphylococcal enterotoxin B antibody advantageously results in reduced incidence and/or amelioration of one or more symptoms of staphylococcal sepsis including, for example, fever, chills, shaking, rapid heartbeat, rapid breathing, low blood pressure, confusion, disorientation, agitation, dizziness, decreased urination, rash, and joint pain.

A subject suffering from or at risk for staphylococcal sepsis can be treated with a Staphylococcal enterotoxin B antibody. A subject suitable for Staphylococcal enterotoxin B antibody therapy is selected using clinical criteria and prognostic indicators of staphylococcal sepsis that are well known in the art. Assessment of staphylococcal sepsis severity may be performed based on tests known in the art, including blood culture to detect bacteria, sampling of sputum, urine, spinal fluid and/or abscess contents to detect bacteria, chest X-ray, and CT scan. In some embodiments, ameliorating, controlling, reducing incidence of, or delaying the development or progression of staphylococcal sepsis and/or symptoms of staphylococcal sepsis is measured by blood culture analysis.

In some embodiments, therapeutic administration of the Staphylococcal enterotoxin B antibody advantageously results in reduced incidence and/or amelioration of one or more symptoms of staphylococcal gastroenteritis including, for example, fever, headache, chills, myalgias, malaise, nausea, vomiting, intestinal cramping and/or diarrhea.

A subject suffering from or at risk for staphylococcal gastroenteritis can be treated with a Staphylococcal enterotoxin B antibody. A subject suitable for Staphylococcal enterotoxin B antibody therapy is selected using clinical criteria and prognostic indicators of staphylococcal gastroenteritis that are well known in the art. Assessment of staphylococcal gastroenteritis severity may be performed based on tests known in the art, including, for example, chest radiography, abdominal radiography, pulse oximetry, complete blood count, serum electrolyte count, and tests to specifically identify the Staphylococcal toxin. In some embodiments, ameliorating, controlling, reducing incidence of, or delaying the development or progression of staphylococcal gastroenteritis is measured by a combination of physical signs and the patient's physiologic condition.

With respect to all methods described herein, reference to anti-Staphylococcal enterotoxin B antibodies, or equivalent, also includes compositions comprising one or more additional agents. These compositions may further comprise suitable excipients, such as pharmaceutically acceptable excipients including buffers, which are well known in the art. The present invention can be used alone or in combination with other methods of treatment.

In an embodiment, with respect to all methods described herein, the reference to anti-Staphylococcal enterotoxin B antibodies, or equivalent, also includes compositions comprising Vancomycin and/or co-administered with a course of Vancomycin.

The Staphylococcal enterotoxin B antibody can be administered to a subject via any suitable route. It should be apparent to a person skilled in the art that the examples described herein are not intended to be limiting but to be illustrative of the techniques available. Accordingly, in some embodiments, the Staphylococcal enterotoxin B antibody is administered to a subject in accord with known methods, such as intravenous administration, e.g., as a bolus or by continuous infusion over a period of time, by intramuscular, intraperitoneal, intracerebrospinal, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, Staphylococcal enterotoxin B antibody can be aerosolized using a fluorocarbon formulation and a metered dose inhaler, or inhaled as a lyophilized and milled powder.

In some embodiments, a Staphylococcal enterotoxin B antibody is administered via site-specific or targeted local delivery techniques. Examples of site-specific or targeted local delivery techniques include various implantable depot sources of the Staphylococcal enterotoxin B antibody or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of a Staphylococcal enterotoxin B antibody may be used for administration. In some embodiments, the Staphylococcal enterotoxin B antibody may be administered neat. In some embodiments, Staphylococcal enterotoxin B antibody and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular subject and that subject's medical history.

A Staphylococcal enterotoxin B antibody can be administered using any suitable method, including by injection (e.g., intraperitoneally, intravenously, subcutaneously, intramuscularly, etc.). Staphylococcal enterotoxin B antibodies can also be administered topically or via inhalation, as described herein. Generally, for administration of Staphylococcal enterotoxin B antibodies, an initial candidate dosage can be about 2 mg/kg. For the purpose of the present invention, a typical daily dosage might range from about any of 3 μg/kg to 30 μg/kg to 300 μg/kg to 3 mg/kg, to 30 mg/kg, to 100 mg/kg or more, depending on the factors mentioned above. For example, dosage of about 1 mg/kg, about 2.5 mg/kg, about 5 mg/kg, about 10 mg/kg, and about 25 mg/kg may be used. For repeated administrations over several days or longer, depending on the condition, the treatment is sustained until a desired suppression of symptoms occurs or until sufficient therapeutic levels are achieved, for example, to reduce symptoms associated with staphylococcal disease. The progress of this therapy is easily monitored by conventional techniques and assays. The dosing regimen (including the Staphylococcal enterotoxin B antibody used) can vary over time.

For the purpose of the present invention, the appropriate dosage of an Staphylococcal enterotoxin B antibody will depend on the Staphylococcal enterotoxin B antibody (or compositions thereof) employed, the type and severity of symptoms to be treated, whether the agent is administered for preventive or therapeutic purposes, previous therapy, the patient's clinical history and response to the agent, the amount of S. aureus present at the disease site, the patient's clearance rate for the administered agent, and the discretion of the attending physician. Typically, the clinician will administer an anti-Staphylococcal enterotoxin B antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of Staphylococcal enterotoxin B antibodies may be appropriate. Various formulations and devices for achieving sustained release are known in the art.

In one embodiment, dosages for an antibody may be determined empirically in subjects who have been given one or more administration(s) of an antibody. Subjects are given incremental dosages of a Staphylococcal enterotoxin B antibody. To assess efficacy, an indicator of the disease can be followed.

Administration of an Staphylococcal enterotoxin B antibody in accordance with the method in the present invention can be continuous or intermittent, depending, for example, upon the recipient's physiological condition, whether the purpose of the administration is therapeutic or prophylactic, and other factors known to skilled practitioners. The administration of a Staphylococcal enterotoxin B antibody may be essentially continuous over a preselected period of time or may be in a series of spaced doses.

In some embodiments, more than one Staphylococcal enterotoxin B antibody may be present. At least one, at least two, at least three, at least four, at least five different, or more antibodies can be present. Generally, those Staphylococcal enterotoxin B antibodies may have complementary activities that do not adversely affect each other. An Staphylococcal enterotoxin B antibody can also be used in conjunction with other antibodies to S. aureus, and/or other therapies. A Staphylococcal enterotoxin B antibody can also be used in conjunction with other agents that serve to enhance and/or complement the effectiveness of the agents.

In some embodiments, the Staphylococcal enterotoxin B antibody may be administered in combination with the administration of traditional therapies. These include, but are not limited to, the administration of antibiotics such as methicillin, linezolid, torezolid, eperezolid, posizolid, radezolid, streptomycin, neomycin, kanamycin, spectinomycin, p aromomyc in, gentamycin, verdamicin, astromic in, ciprofloxacin, daptomycin, doxycycline, chlortetracycline, clomocycline, demeclocycline, lymecycline, meclocycline, metacycline, minocycline, oxytetracycline, penimepicycline, rolitetracycline, tetracycline, tigecycline, chloramphenicol, azidamfenicol, thiamphenicol, florfenicol, retapamulin, tiamulin, valnemulin, erythromycin, azithromycin, spiramycin, midecamycin, oleandomycin, roxithromycin, josamycin, troleandomycin, clarithromycin, miocamycin, rokitamycin, dirithromycin, flurithromycin, ketolide (e.g., telithromycin, cethromycin, and solithromycin), clindamycin, lincomycin, pristinamycin, ampicillin, oxacillin, fusidic acid, virginiamycin, quinupristin, dalfopristin, vancomycin, penicillin, trimethoprim, sulfamethoxazole, or various combinations of antibiotics. Administration of Staphylococcal enterotoxin B antibodies to an subject may be used in combination with the administration of antivirulence agents, such as the RNAIII-inhibiting heptapeptide (RIP).

In some embodiments, a Staphylococcal enterotoxin B antibody is used in conjunction with antibacterial and/or antivirulence agent treatment. Alternatively, the antibody therapy may precede or follow the other agent treatment by intervals ranging from minutes to weeks. In embodiments where the other agents and/or a proteins or polynucleotides are administered separately, one would generally ensure that a significant period of time did not expire between each delivery, such that the agent and the antibody composition of the present invention would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations. In an embodiment, the agent is Vancomycin.

Therapeutic formulations of the Staphylococcal enterotoxin B antibody used in accordance with the present invention are prepared for storage by mixing an antibody having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the Staphylococcal enterotoxin B antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic Staphylococcal enterotoxin B antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-Staphylococcal enterotoxin B antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

As used herein, “treating” refers to preventing, treating, reducing the severity of, and/or ameliorating a staphylococcal disease in an subject. Unless otherwise indicated, it also refers to preventing or delaying mortality attributable to the disease, decreasing the number of bacteria recoverable from the affected site (e.g., lung, skin, breast, etc.), limiting the pathological lesions to focal sites, decreasing the extent of damage from the disease, decreasing the duration of the disease, and/or reducing the number, extent, or duration of symptoms related to the disease.

The term “subject” is intended to include mammals, e.g., humans, dogs, cows, horses, pigs, sheep, goats, cats, mice, rabbits, rats, and also includes avians. In specific embodiments of the invention, the subject is a human.

In an embodiment, the antibodies, fragments or scFvs of the invention specifically bind SEB as set forth in SEQ ID NO:30. In an embodiment, the antibodies, fragments or scFvs of the invention specifically bind SEB as set forth in SEQ ID NO:26.

In an embodiment, the SEB has the sequence:

| (SEQ ID NO: 30) | |
|---|---|
| ESQPDPKPDE LHKSSKFTGL MENMKVLYDD NHVSAINVKS IDQFLYFDLI YSIKDTKLGN | 60 |
| YDNVRVEFKN KDLADKYKDK YVDVFGANYY YQCYFSKKTN DINSHQTDKR KTCMYGGVTE | 120 |
| HNGNQLDKYR SITVRVFEDG KNLLSFDVQT NKKKVTAQEL DYLTRHYLVK NKKLYEFNNS | 180 |
| PYETGYIKFI ENENSFWYDM MPAPGDKFDQ SKYLMMYNDN KMVDSKDVKI EVYLTTKKK. | 239 |

In an embodiment, the SEB from MRSA has the sequence:

| (SEQ ID NO: 26) | |
|---|---|
| ESQPDPKPDE LHKSSKFTGL MENMKVLYDD NHVSAINVKS IDQFLYFDLI YSIKDTKLGN | 60 |
| YDNVRVEFKN KDLADKYKDK YVDVFGANYY YQCYFSKKTN DINSHQTDKR KTCMYGGVTE | 120 |
| HNGNQLDKYR SITVRVFEDG KNLLSFDVQT NKKKVTAQEL DYLTRHYLVK NKKLYEFNNS | 180 |
| PYETGYIKFI ENENSFWYDM MPAPGDKFDQ SKYLMMYNDN KMVDSKDVKI EVYLYDKEK | 239 |

Accordingly, the invention provides any of the following, or compositions (including pharmaceutical compositions) comprising, an antibody having a partial light chain sequence and a partial heavy chain sequence as found in Table 1, or variants thereof. In Table 1, the bold and underlined sequences are CDR sequences according to AbM.

The invention also provides CDR portions of antibodies to Staphylococcal enterotoxin B. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of any one of the heavy chain variable regions shown in Table 1. In some embodiments, the antibody comprises three CDRs of any one of the light chain variable regions shown in Table 1. In some embodiments, the antibody comprises three CDRs of any one of the heavy chain variable regions shown in Table 1, and three CDRs of any one of the light chain variable regions shown in Table 1.

Table 2 provides examples of CDR sequences of anti-Staphylococcal enterotoxin B antibodies provided herein. Examples of CDR sequences are described in Example 3 below.

The invention also provides methods of generating, selecting, and making Staphylococcal enterotoxin B antibodies. The antibodies of this invention can be made by procedures known in the art. In some embodiments, antibodies may be made recombinantly and expressed using any method known in the art.

In some embodiments, antibodies may be prepared and selected by phage display technology. See, for example, U.S. Pat. Nos. 5,565,332; 5,580,717; 5,733,743; and 6,265,150; and Winter et al., Annu. Rev. Immunol. 12:433-455, 1994. Alternatively, the phage display technology (McCafferty et al., Nature 348:552-553, 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3:564-571, 1993. Several sources of V-gene segments can be used for phage display. Clackson et al., Nature 352:624-628, 1991, isolated a diverse array of anti-oxazolone antibodies from a small random combinatorial library of V genes derived from the spleens of immunized mice. A repertoire of V genes from human donors can be constructed and antibodies to a diverse array of antigens (including self-antigens) can be isolated essentially following the techniques described by Mark et al., J. Mol. Biol. 222:581-597, 1991, or Griffith et al., EMBO J. 12:725-734, 1993. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” (Marks et al., Bio/Technol. 10:779-783, 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266, 1993. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., 1975, Nature 256:495-497 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the Staphylococcal enterotoxin B monoclonal antibodies of the subject invention. The hybridomas or other immortalized B-cells are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for Staphylococcal enterotoxin B, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen Immunization of a host animal with an Staphylococcal enterotoxin B polypeptide, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N═C═NR, where R and R1 are different alkyl groups, can yield a population of antibodies (e.g., monoclonal antibodies).

If desired, the Staphylococcal enterotoxin B antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756, 2001; Lonberg, N. and D. Huszar Int. Rev. Immunol 13:65, 1995; and Pollock, et al., J Immunol Methods 231:147, 1999. Methods for making derivatives of antibodies, e.g., domain, single chain, etc. are known in the art.

Immunoassays and flow cytometry sorting techniques such as fluorescence activated cell sorting (FACS) can also be employed to isolate antibodies that are specific for Staphylococcal enterotoxin B.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851, 1984, or by covalently joining to the immunoglobulin coding sequence all or part of the coding sequence for a non-immunoglobulin polypeptide. In that manner, “chimeric” or “hybrid” antibodies are prepared that have the binding specificity of an Staphylococcal enterotoxin B monoclonal antibody herein.

Antibody fragments can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In some embodiments, a polynucleotide comprises a sequence encoding the heavy chain and/or the light chain variable regions of antibody DF1.1, DF1, DF2, DF3, DF4, DF5, DF6, DF7, DFB, DF9, DF10, DF11, DF12, DF13, DF14, DF15, DF16, DF17, DF18, DF19 or DF20. The sequence encoding the antibody of interest may be maintained in a vector in a host cell and the host cell can then be expanded and frozen for future use. Vectors (including expression vectors) and host cells are further described herein.

The invention includes affinity matured embodiments. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al., 1992, J. Mol. Biol., 226:889-896; and PCT Publication No. WO2004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using, for example, Biacore™ surface plasmon resonance analysis, which detects differences in binding affinity of about 2-fold or greater, Kinexa® Biosensor, scintillation proximity assays, ELISA, ORIGEN® immunoassay, fluorescence quenching, fluorescence transfer, and/or yeast display. Binding affinity may also be screened using a suitable bioassay. Biacore™ is particularly useful when the starting antibody already binds with a relatively high affinity, for example a KD of about 10 nM or lower.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., 1993, Gene 137(1):109-18.

The CDR may be heavy chain variable region (VH) CDR3 and/or light chain variable region (VL) CDR3. The CDR may be one or more of VH CDR1, VH CDR2, VH CDR3, VL CDR1, VL CDR2, and/or VL CDR3. The CDR may be a Kabat CDR, a Chothia CDR, an extended CDR, an AbM CDR, a contact CDR, or a conformational CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using Kinexa™ biosensor analysis, and selection using any method known in the art for selection, including phage display, yeast display, and ribosome display.

To express the Staphylococcal enterotoxin B antibodies of the present invention, DNA fragments encoding VH and VL regions can first be obtained using any of the methods described above. Various modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

The invention encompasses modifications to the variable regions shown in Table 1 and the CDRs shown in Table 2. For example, the invention includes antibodies comprising functionally equivalent variable regions and CDRs which do not significantly affect their properties as well as variants which have enhanced or decreased activity and/or affinity. For example, the amino acid sequence may be mutated to obtain an antibody with the desired binding affinity to Staphylococcal enterotoxin B. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 3 under the heading of “conservative substitutions.” If such substitutions result in a change in biological activity, then more substantial changes, denominated “exemplary substitutions” in Table 3, or as further described below in reference to amino acid classes, may be introduced and the products screened.

TABLE 3
Amino Acid Substitutions

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

The antibodies may also be modified, e.g. in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the KD of the antibody for Staphylococcal enterotoxin B, to increase or decrease koff, or to alter the binding specificity of the antibody. Techniques in site-directed mutagenesis are well-known in the art. See, e.g., Sambrook et al., and Ausubel et al., supra.

A modification or mutation may also be made in a framework region or constant region to increase the half-life of a Staphylococcal enterotoxin B antibody. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. According to the invention, a single antibody may have mutations in any one or more of the CDRs or framework regions of the variable domain or in the constant region.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g., Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Application No. PCT/GB99/01441; and/or UK Patent Application No. 9809951.8.

In some embodiments, an antibody constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In some embodiments, the constant region is modified as described in Eur. J. Immunol., 1999, 29:2613-2624; PCT Application No. PCT/GB99/01441; and/or UK Patent Application No. 9809951.8. In such embodiments, the Fc can be human IgG2 or human IgG4. The Fc can be human IgG2 containing the mutation A330P331 to S330S331 (IgG2Aa), in which the amino acid residues are numbered with reference to the wild type IgG2 sequence. Eur. J. Immunol., 1999, 29:2613-2624. In some embodiments, the antibody comprises a constant region of IgG4 comprising the following mutations (Armour et al., 2003, Molecular Immunology 40 585-593): E233/F234/L235 to P233/V234/A235 (IgG4Δc), in which the numbering is with reference to wild type IgG4. In yet another embodiment, the Fc is human IgG4 E233F234L235 to P233V234A235 with deletion G236 (IgG4Δb). In another embodiment the Fc is any human IgG4 Fc (IgG4, IgG4Δb or IgG4Δc) containing hinge stabilizing mutation 5228 to P228 (Aalberse et al., 2002, Immunology 105, 9-19).

In some embodiments, the portion of the humanized immunoglobulin or immunoglobulin chain which is of human origin (the human portion) can be derived from any suitable human immunoglobulin or immunoglobulin chain. For example, a human constant region or portion thereof, if present, can be derived from the κ or λ light chains, and/or the γ (e.g., γ1, γ2, γ3, γ4), μ, α (e.g., α1, α2), δ or ε heavy chains of human antibodies, including allelic variants. A particular constant region, such as IgG2 or IgG4, variants or portions thereof can be selected to tailor effector function. For example, a mutated constant region can be incorporated into a fusion protein to minimize binding to Fc receptors and/or ability to fix complement (see e.g., Winter et al., U.S. Pat. Nos. 5,648,260 and 5,624,821; GB 2,209,757 B; Morrison et al., WO 89/07142; Morgan et al., WO 94/29351, Dec. 22, 1994). In some embodiments, the humanized heavy chain constant domain is of the IgG1 isotype and comprises the triple mutations (TM) L234A/L235A/G237A such that the effector function of the Fc domain of the antibody is reduced. The numbering of the residues in the Fc region is that of the EU index as in Kabat. Kabat et al., Sequences of Proteins of Immunological Interest, 5th Ed. Public Health Service, National Institutes of Health, Bethesda, Md., 1991.

In some embodiments, the antibody comprises a human heavy chain IgG2 constant region comprising the following mutations: A330P331 to S330S331 (amino acid numbering with reference to the wild type IgG2 sequence). Eur. J. Immunol., 1999, 29:2613-2624. In still other embodiments, the constant region is aglycosylated for N-linked glycosylation. In some embodiments, the constant region is aglycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the antibody comprises a modified constant region that has increased binding affinity for FcRn and/or an increased serum half-life as compared with the unmodified antibody.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant region of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of a Staphylococcal enterotoxin B antibody of the invention can be cleaved. In various embodiments of the invention, the heavy and light chains of the Staphylococcal enterotoxin B antibodies may optionally include a signal sequence.

Once DNA fragments encoding the VH and VL segments of the present invention are obtained, these DNA fragments can be further manipulated by standard recombinant DNA techniques, for example to convert the variable region genes to full-length antibody chain genes, to Fab fragment genes, or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG2 constant region. The IgG constant region sequence can be any of the various alleles or allotypes known to occur among different individuals, such as Gm(1), Gm(2), Gm(3), and Gm(17). These allotypes represent naturally occurring amino acid substitution in the IgG1 constant regions. For a Fab fragment heavy chain gene, the VH-encoding DNA can be operatively linked to another DNA molecule encoding only the heavy chain CH1 constant region. The CH1 heavy chain constant region may be derived from any of the heavy chain genes.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. The kappa constant region may be any of the various alleles known to occur among different individuals, such as Inv(1), Inv(2), and Inv(3). The lambda constant region may be derived from any of the three lambda genes.

To create a scFv gene, the VH- and VL-encoding DNA fragments are operatively linked to another fragment encoding a flexible linker such that the VH and VL sequences can be expressed as a contiguous single-chain protein, with the VL and VH regions joined by the flexible linker (See e.g., Bird et al., 1988, Science 242:423-426; Huston et al., 1988, Proc. Natl. Acad. Sci. USA 85:5879-5883; McCafferty et al., 1990, Nature 348:552-554. An example of a linking peptide is (GGGGS)3 (SEQ ID NO: 18), which bridges approximately 3.5 nm between the carboxy terminus of one variable region and the amino terminus of the other variable region. Linkers of other sequences have been designed and used (Bird et al., 1988, supra). Linkers can in turn be modified for additional functions, such as attachment of drugs or attachment to solid supports. The single chain antibody may be monovalent, if only a single VH and VL pair is used, bivalent, if two VH and VL are used, or polyvalent, if more than two VH and VL are used. Bispecific or polyvalent antibodies may be generated that bind specifically to Staphylococcal enterotoxin B and to another molecule. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies, are also encompassed. Diabodies are bivalent, bispecific antibodies in which VH and VL are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., 1993, Proc. Natl. Acad Sci. USA 90:6444-6448; Poljak, R. J., et al., 1994, Structure 2:1121-1123).

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

The invention also encompasses fusion proteins comprising one or more fragments or regions from the antibodies disclosed herein. In some embodiments, a fusion antibody may be made that comprises all or a portion of a Staphylococcal enterotoxin B antibody of the invention linked to another polypeptide. In another embodiment, only the variable domains of the Staphylococcal enterotoxin B antibody are linked to the polypeptide. In another embodiment, the VH domain of a Staphylococcal enterotoxin B antibody is linked to a first polypeptide, while the VL domain of an Staphylococcal enterotoxin B antibody is linked to a second polypeptide that associates with the first polypeptide in a manner such that the VH and VL domains can interact with one another to form an antigen binding site. In another preferred embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

A fusion polypeptide can be created by methods known in the art, for example, synthetically or recombinantly. Typically, the fusion proteins of this invention are made by preparing an expressing a polynucleotide encoding them using recombinant methods described herein, although they may also be prepared by other means known in the art, including, for example, chemical synthesis.

In other embodiments, other modified antibodies may be prepared using Staphylococcal enterotoxin B antibody encoding nucleic acid molecules. For instance, “Kappa bodies” (Ill et al., 1997, Protein Eng. 10:949-57), “Minibodies” (Martin et al., 1994, EMBO J. 13:5303-9), “Diabodies” (Holliger et al., supra), or “Janusins” (Traunecker et al., 1991, EMBO J. 10:3655-3659 and Traunecker et al., 1992, Int. J. Cancer (Suppl.) 7:51-52) may be prepared using standard molecular biological techniques following the teachings of the specification.

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). For example, bispecific antibodies or antigen-binding fragments can be produced by fusion of hybridomas or linking of Fab′ fragments. See, e.g., Songsivilai & Lachmann, 1990, Clin. Exp. Immunol. 79:315-321, Kostelny et al., 1992, J. Immunol. 148:1547-1553. Traditionally, the recombinant production of bispecific antibodies was based on the coexpression of two immunoglobulin heavy chain-light chain pairs, with the two heavy chains having different specificities (Millstein and Cuello, 1983, Nature 305, 537-539). In addition, bispecific antibodies may be formed as “diabodies” or “Janusins.” In some embodiments, the bispecific antibody binds to two different epitopes of Staphylococcal enterotoxin B. In some embodiments, the modified antibodies described above are prepared using one or more of the variable domains or CDR regions from a Staphylococcal enterotoxin B antibody provided herein.

According to one approach to making bispecific antibodies, antibody variable domains with the desired binding specificities (antibody-antigen combining sites) are fused to immunoglobulin constant region sequences. The fusion preferably is with an immunoglobulin heavy chain constant region, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

This invention also provides compositions comprising antibodies conjugated (for example, linked) to an agent that facilitate coupling to a solid support (such as biotin or avidin). For simplicity, reference will be made generally to antibodies with the understanding that these methods apply to any of the Staphylococcal enterotoxin B binding and/or antagonist embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation. In some embodiments, the carrier comprises a moiety that targets the lung, heart, or heart valve.

An antibody or polypeptide of this invention may be linked to a labeling agent such as a fluorescent molecule, a radioactive molecule or any others labels known in the art. Labels are known in the art which generally provide (either directly or indirectly) a signal.

Polynucleotides, vectors, and host cells: The invention also provides polynucleotides encoding any of the antibodies, including antibody fragments and modified antibodies described herein, such as, e.g., antibodies having impaired effector function. In another aspect, the invention provides a method of making any of the polynucleotides described herein. Polynucleotides can be made and expressed by procedures known in the art. Accordingly, the invention provides polynucleotides or compositions, including pharmaceutical compositions, comprising polynucleotides, encoding any combination of the heavy and light chains of the following: the antibodies or antigen-binding fragments thereof, hu20B1 VH v1.0, hu20B1 VL v1.1 (ATCC accession number PTA-13616), hu20B1 VL v1.4; hu20B1 VL v1.6; hu20B1 VL v1.10; hu20B1 VH v1.0; hu20B1 VH v1.1, hu20B1 VH v1.4 (ATCC accession number PTA-13617), hu20B1 VH v1.6 (ATCC accession number PTA-13618), hu20B1 VH v1.10, hu20B1v1.0/1.0, hu20B1v1.1/1.1; hu20B1v1.4/1.1; hu20B1v1.6/1.1; and hu20B1v1.10/1.1; or any fragment or part thereof having the ability to antagonize Staphylococcal enterotoxin B.

The invention encompasses an antibody, or antigen-binding fragment thereof, comprising the three CDRs of the light chain variable domain amino acid sequence encoded by the polynucleotide insert of the vector deposited with the ATCC on Mar. 13, 2013, as hu20B1 VL v1.1 (ATCC Acc. No. PTA-13616), and three CDRs of the heavy chain variable domain amino acid sequence encoded by the polynucleotide insert of the vector deposited with the ATCC on Mar. 13, 2013, as hu20B1 VH v1.4 (ATCC Acc. No. PTA-13617) or the three CDRs of the heavy chain variable domain amino acid sequence encoded by the polynucleotide insert of the vector deposited with the ATCC on Mar. 13, 2013, as hu20B1 VH v1.6 (ATCC Acc. No. PTA-13618).

The invention encompasses an antibody, or antigen-binding fragment thereof, comprising the light chain variable domain amino acid sequence encoded by the polynucleotide insert of the vector deposited with the ATCC on Mar. 13, 2013, as hu20B1 VL v1.1 (ATCC Acc. No. PTA-13616), and the heavy chain variable domain amino acid sequence encoded by the polynucleotide insert of the vector deposited with the ATCC on Mar. 13, 2013, as hu20B1 VH v1.4 (ATCC Acc. No. PTA-13617) or the heavy chain variable domain amino acid sequence encoded by the polynucleotide insert of the vector deposited with the ATCC on Mar. 13, 2013, as hu20B1 VH v1.6 (ATCC Acc. No. PTA-13618).

The invention encompasses an antibody, or antigen-binding fragment thereof, comprising the light chain variable domain amino acid sequence encoded by the polynucleotide insert of the vector deposited with the ATCC on Mar. 13, 2013, as hu20B1 VL v1.1 (ATCC Acc. No. PTA-13616), and the heavy chain variable domain amino acid sequence encoded by the polynucleotide insert of the vector deposited with the ATCC on Mar. 13, 2013, as hu20B1 VH v1.4 (ATCC Acc. No. PTA-13617).

The invention also encompasses an antibody, or antigen-binding fragment thereof, comprising the light chain variable domain amino acid sequence encoded by the polynucleotide insert of the vector deposited with the ATCC on Mar. 13, 2013, as hu20B1 VL v1.1 (ATCC Acc. No. PTA-13616), and the heavy chain variable domain amino acid sequence encoded by the polynucleotide insert of the vector deposited with the ATCC on Mar. 13, 2013, as hu20B1 VH v1.6 (ATCC Acc. No. PTA-13618).

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, Co1E1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to Staphylococcal enterotoxin B or an Staphylococcal enterotoxin B domain is affected by an immunoassay or FACS. A cell overexpressing the antibody or protein of interest can be identified.

An expression vector can be used to direct expression of a Staphylococcal enterotoxin B antibody. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventricle, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J. A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338. Therapeutic compositions containing a polynucleotide are administered in a range of about 100 ng to about 200 mg of DNA for local administration in a gene therapy protocol. Concentration ranges of about 500 ng to about 50 mg, about 1 ng to about 2 mg, about 5 ng to about 500 ng, and about 20 ng to about 100 ng of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura, Human Gene Therapy, 1994, 5:845; Connelly, Human Gene Therapy, 1995, 1:185; and Kaplitt, Nature Genetics, 1994, 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581.

The invention also provides pharmaceutical compositions comprising an effective amount of a Staphylococcal enterotoxin B antibody described herein. Examples of such compositions, as well as how to formulate, are also described herein. In some embodiments, the composition comprises one or more Staphylococcal enterotoxin B antibodies. In other embodiments, the Staphylococcal enterotoxin B antibody recognizes Staphylococcal enterotoxin B. In other embodiments, the Staphylococcal enterotoxin B antibody is a human antibody. In other embodiments, the Staphylococcal enterotoxin B antibody is a humanized antibody. In some embodiments, the Staphylococcal enterotoxin B antibody comprises a constant region that is capable of triggering a desired immune response, such as antibody-mediated lysis or ADCC. In other embodiments, the Staphylococcal enterotoxin B antibody comprises a constant region that does not trigger an unwanted or undesirable immune response, such as antibody-mediated lysis or ADCC. In other embodiments, the Staphylococcal enterotoxin B antibody comprises one or more CDR(s) of the antibody (such as one, two, three, four, five, or, in some embodiments, all six CDRs).

It is understood that the compositions can comprise more than one Staphylococcal enterotoxin B antibody (e.g., a mixture of Staphylococcal enterotoxin B antibodies that recognize different epitopes of Staphylococcal enterotoxin B). Other exemplary compositions comprise more than one Staphylococcal enterotoxin B antibody that recognize the same epitope(s), or different species of Staphylococcal enterotoxin B antibodies that bind to different epitopes of Staphylococcal enterotoxin B. In some embodiments, the compositions comprise a mixture of Staphylococcal enterotoxin B antibodies that recognize different variants of Staphylococcal enterotoxin B.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The Staphylococcal enterotoxin B antibody and compositions thereof can also be used in conjunction with other agents that serve to enhance and/or complement the effectiveness of the agents.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. The preceding description applies to all polynucleotides referred to herein, including RNA and DNA.

The invention also provides compositions, including pharmaceutical compositions, comprising any of the polynucleotides of the invention. In some embodiments, the composition comprises an expression vector comprising a polynucleotide encoding the antibody as described herein. In other embodiment, the composition comprises an expression vector comprising a polynucleotide encoding any of the antibodies described herein. In still other embodiments, the composition comprises either or both of the polynucleotides shown in SEQ ID NO: 22 and SEQ ID NO: 28 below, or either or both of the polynucleotides shown in SEQ ID NO: 24 and SEQ ID NO: 28 below:

hu20B1 VH v1.4 Heavy Chain Variable Region

(SEQ ID NO: 22)
GAGGTGCAGCTGGTGGAGTCTGGGGGAGGCTTGGTCCAGCCTGGGGGGTC
CCTGAGACTCTCCTGTGCAGCCTCTGGGTATATCTTCACAATTGCGGGAA
TACAGTGGGTCCGCCAGGCTCCAGGGAAGGGGCTGGAGTGGGTGGGATGG
ATAAACACCCATTCTGGAGTGCCAGAATATGCAGAAGAGTTCAAGGGACG
ATTCACCATCTCCCTGGACAACGCCAAGAACTCAGCATATCTGCAAATGA
ACAGCCTGAGAGCCGAGGACACGGCTGTGTATTACTGTGCGAGAATCTAC
TATGGTAACAACGGGGGTGTTATGGACTATTGGGGCCAAGGAACCCTGGT
CACCGTCTCCTCA

hu20B1 VH v1.6 Heavy Chain Variable Region

(SEQ ID NO: 24)
GAGGTGCAGCTGGTGGAGTCTGGGGGAGGCTTGGTCCAGCCTGGGGGGTC
CCTGAGACTCTCCTGTGCAGCCTCTGGGTATATCTTCACAATTGCGGGAA
TACAGTGGGTCCGCCAGGCTCCAGGGAAGGGGCTGGAGTGGGTGGGATGG
ATAAACACCCATTCTGGAGTGCCAGAATATGCAGAAGAGTTCAAGGGACG
ATTCACCTTCTCCCTGGACAACGCCAAGAACTCAGCATATCTGCAAATGA
ACAGCCTGAGAGCCGAGGACACGGCTGTGTATTACTGTGCGAGAATCTAC
TATGGTAACAACGGGGGTGTTATGGACTATTGGGGCCAAGGAACCCTGGT
CACCGTCTCCTCA

hu20B1 VL v1.1 Light Chain Variable Region

(SEQ ID NO: 28)
GACATCCAGATGACCCAGTCTCCATCCTCCCTGTCTGCATCTGTAGGAGA
CAGAGTCACCATCACTTGCCGGGCAAGTCAGGAAATTAGTGATTACTTAA
CCTGGCTCCAGCAGAAACCAGGGAAAGCCATCAAGCGCCTGATCTATGTC
GCATCCAGTTTAGATTCTGGGGTCCCATCAAGGTTCAGTGGCAGTCGTTC
TGGGACAGATTTCACTCTCACCATCAGCAGTCTGCAACCTGAAGATTTTG
CAACTTACTACTGTCTACAATATGCTAATTATCCGTGGACGTTCGGCGGA
GGGACCAAGGTGGAGATCAAA

The invention also provides kits comprising any or all of the antibodies described herein. Kits of the invention include one or more containers comprising an Staphylococcal enterotoxin B antibody described herein and instructions for use in accordance with any of the methods of the invention described herein. Generally, these instructions comprise a description of administration of the Staphylococcal enterotoxin B antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In some embodiments, the kit comprises Vancomycin.

In some embodiments, the antibody is a human antibody. In some embodiments, the antibody is a humanized antibody. In some embodiments, the antibody is a monoclonal antibody. The instructions relating to the use of a Staphylococcal enterotoxin B antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an Staphylococcal enterotoxin B antibody. The container may further comprise a second pharmaceutically active agent.

Kits may optionally provide additional components such as buffers and interpretive information. Normally, the kit comprises a container and a label or package insert(s) on or associated with the container.

The invention also provides diagnostic kits comprising any or all of the antibodies described herein. The diagnostic kits are useful for, for example, detecting the presence of Staphylococcal enterotoxin B in a sample. In some embodiments, a diagnostic kit can be used to identify an individual with a latent S. aureus infection that may put them at risk of developing an invasive form of infection. In some embodiments, a diagnostic kit can be used to determine whether an individual is at risk for a staphylococcal disease. In some embodiments, a diagnostic kit can be used to detect the presence of Staphylococcal enterotoxin B in an individual suspected of having a staphylococcal disease.

Diagnostic kits of the invention include one or more containers comprising an Staphylococcal enterotoxin B antibody described herein and instructions for use in accordance with any of the methods of the invention described herein. Generally, these instructions comprise a description of use of the Staphylococcal enterotoxin B antibody to detect the presence of Staphylococcal enterotoxin B in individuals at risk for, or suspected of having, staphylococcal disease. In some embodiments, an exemplary diagnostic kit can be configured to contain reagents such as, for example, a Staphylococcal enterotoxin B antibody, a negative control sample, a positive control sample, and directions for using the kit.

### Biological Deposit:

Representative materials of the present invention were deposited in the American Type Culture Collection, 10801 University Boulevard, Manassas, Va. 20110-2209, USA, on Mar. 13, 2013: a plasmid vector DNA comprising an insert encoding the hu20B1 VL v1.1 (ATCC accession number PTA-13616); a plasmid vector DNA comprising an insert encoding the hu20B1 VH v1.4 (ATCC accession number PTA-13617); and a plasmid vector DNA comprising an insert encoding the hu20B1 VH v1.6 having (ATCC accession number PTA-13618). The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The phrase “and/or” as used herein, with option A and/or option B for example, encompasses the embodiments of (i) option A, (ii) option B, and (iii) option A plus option B.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group subjectly and all possible subgroups of the main group, but also the main group absent one or more of the group members. The present invention also envisages the explicit exclusion of one or more of any of the group members in the claimed invention.

All combinations of the various elements described herein are within the scope of the invention unless otherwise indicated herein or otherwise clearly contradicted by context.

In the event that one or more of the literature and similar materials incorporated by reference herein differs from or contradicts this application, including but not limited to defined terms, term usage, described techniques, or the like, this application controls.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

In vitro T cell proliferation—PBMC: PBMC from healthy donor was isolated using Ficoll-plaque method and resuspended at a density of 1×10⁶/ml and cultured in RPMI 1640 with 5% human sera. Cells were treated with various dilutions of mAb 20B1 and induced with 25 ng/ml of SEB (Toxin Technology, Florida) and incubated for 72 h. IC₅₀ of inhibition by mAb in T-cell assays was calculated by XLfit software, IDBS, UK.

### Animal Experiments

SEB intoxication model: 6-8 week old female BALB/c mice were obtained from National Cancer Institute (Bethesda, Md.) and protective efficacy of mAbs 1.4/1.1 and 1.6/1.1 was tested for SEB induced lethal shock (SEBILS) as described elsewhere (Varshney et al JBC). Mice were injected intraperitoneal with once with 500 μg of mAb prior to 25 mg of d-galactosamine in PBS, followed by 20 μg of purified SEB (Toxin technology) and observed death for 5 days. Control mice were treated with PBS only. Blood was obtained from retro-orbital bleeding at 2 and 8h post-toxin injection. Serum was separated and concentration of IFN-γ was quantified by mouse-IFN-γ kit (R&D Systems) according to manufacturer's instructions.

Thigh infection model: Thigh infection was performed as described [31] with some modifications under anesthesia by making a small incision into the lateral aspect of the quadriceps muscle. A loop of suture was embedded into the incision to provide a foreign body substrate for bacterial attachment. MRSA strain 38 (1×10⁵ CFU) was suspended in 5 μl PBS and placed with a pipette deep into the muscle tissue via the incision. The skin was closed with clips and mice were observed for 5 days. Mice were treated 24 hr prior to infection with 500 μg Hu-mAb-1.4/1.1, 1.6/1.1, murine mAb 20B1, or PBS. Vancomycin (Hospira Inc.) was given at a dose of 15 mg/kg i.p. once a day for 3 days starting 24 hrs prior to infection with S. aureus. At day 5, blood was collected via retro-orbital puncture and complete blood count was determined with an automatic hematology analyzer (FORCYTE Hematology Analyzer). Mice were then euthanized and abscesses, spleen and groin lymph nodes were excised and plated for CFU. Part of the abscess fluid was used for cytokine analysis and SEB quantification. Multiplex cytokine analysis was performed using Aushon multiplex system (Aushon Biosystems, Inc.) as per manual instructions. Groin lymph nodes were excised and homogeneized. Lymphocytes were quantified by FACS.

Ethics Statement: All animal experiments were carried out with the approval of the Animal Institute Committee (AIC), in accordance with the rules and regulations set forth by the Albert Einstein College of Medicine AIC.

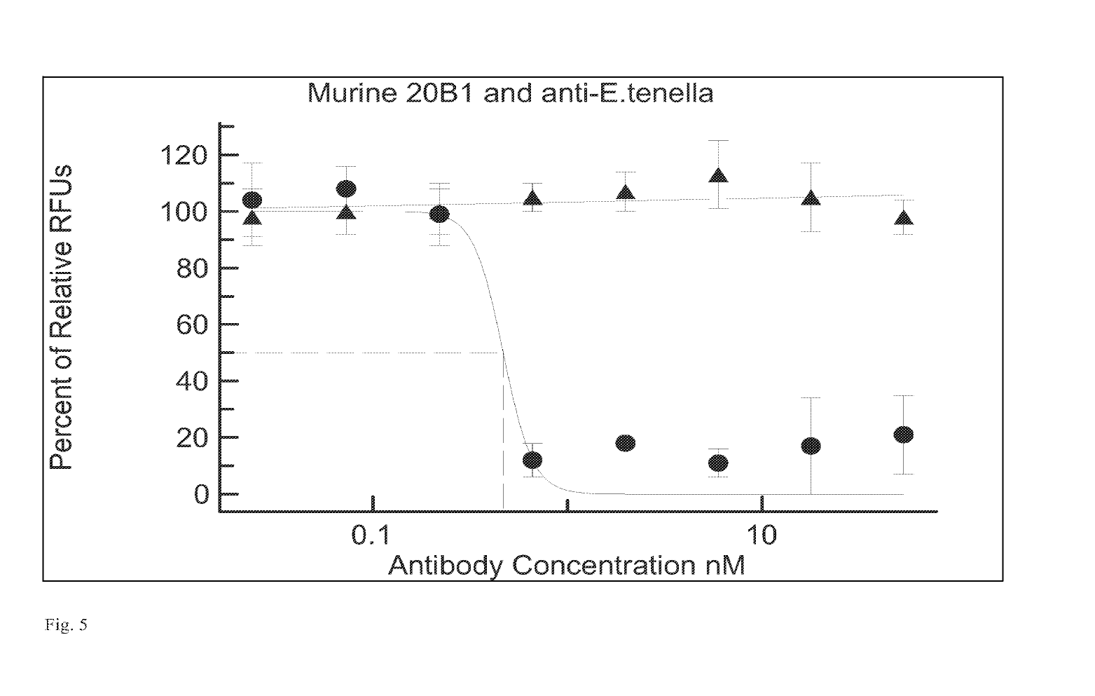

ELISA assay to detect SEB as well as mAb binding to SEB in vivo: Two separate ELISAs were performed to quantify the presence of secreted SEB in the abscess and determine the fraction of unbound SEB left in the abscess. The schematic diagrams of ELISAs are shown in FIG. 5. In the first experiment, the ELISA plate was coated with Goat anti-mouse IgA followed by capture mAb-10F1 (IgA isotype). SEB was detected with mAb-6D3 (IgG2a isotype), which is then bound by alkaline phosphatase (AP) labeled Goat anti-mouse IgG2a Ab (FIG. 5 (schematic ELISA A)). As published elsewhere (Varshney et al JBC 286, 9737-9747 (2011)), mAbs 6D3 and 20B1 bind to distinct non-overlapping epitopes. In the second ELISA (schematic ELISA B) detection is performed with mAb-20B1 (IgG2a isotype switch variant).

TABLE 1
Amino acid sequences of hu20B1 variant heavy and light chain V
domains. Showing CDRs (bold and underline) and back-mutations
in the framework regions (bold).

| Chain | | |
|---|---|---|
| version | | |
| number | VL Domain | VH Domain |
| v1.0 | DIQMTQSPSSLSASVGDRVTITC | EVQLVESGGGLVQPGGSLRLSCAASGYI |
| | RASQEISDYLTWYQQKPGKAPKL | FTIAGIQWVRQAPGKGLEWVAWINTHSG |
| | LIYVASSLDSGVPSRFSGSGSGT | VPEYAEEFKGRFTISRDNAKNSLYLQMN |
| | DFTLTISSLQPEDFATYYCLQYA | SLRAEDTAVYYCARIYYGNNGGVMDYWG |
| | NYPWTFGGGTKVEIK | QGTLVTVSS |
| | (SEQ ID NO: 16) | (SEQ ID NO: 15) |
| v1.1 | DIQMTQSPSSLSASVGDRVTITC | EVQLVESGGGLVQPGGSLRLSCAASGYI |
| | RASQEISDYLTWLQQKPGKAIKR | FTIAGIQWVRQAPGKGLEWVGWINTHSG |
| | LIYVASSLDSGVPSRFSGSRSGT | VPEYAEEFKGRFTISLDNAKNSLYLQMN |
| | DFTLTISSLQPEDFATYYCLQYA | SLRAEDTAVYYCARIYYGNNGGVMDYWG |
| | NYPWTFGGGTKVEIK (SEQ ID | QGTLVTVSS |
| | NO: 27) | (SEQ ID NO: 19) |
| v1.4 | DIQMTQSPSSLSASVGDRVTITC | EVQLVESGGGLVQPGGSLRLSCAASGYI |
| | RASQEISDYLTWLQQKPGKAIKR | FTIAGIQWVRQAPGKGLEWVGWINTHSG |
| | LIYVASSLDSGVPSRFSGSGSGT | VPEYAEEFKGRFTISLDNAKNSAYLQMN |
| | DFTLTISSLQPEDFATYYCLQYA | SLRAEDTAVYYCARIYYGNNGGVMDYWG |
| | NYPWTFGGGTKVEIK (SEQ ID | QGTLVTVSS |
| | NO: 29) | (SEQ ID NO: 21) |
| v1.6 | | EVQLVESGGGLVQPGGSLRLSCAASGYI |
| | | FTIAGIQWVRQAPGKGLEWVGWINTHSG |
| | | VPEYAEEFKGRFTFSLDNAKNSAYLQMN |
| | | SLRAEDTAVYYCARIYYGNNGGVMDYWG |
| | | QGTLVTVSS (SEQ ID NO: 23) |
| v1.10 | | EVQLVESGGGLVQPGGSLRLSCAASGYI |
| | | FTIAGIQWVRQAPGKGLEWVGWINTHSG |
| | | VPEYAEEFKGRFTFSRDNAKNSAYLQMN |
| | | SLRAEDTAVYYCARIYYGNNGGVMDYWG |
| | | QGTLVTVSS (SEQ ID NO: 25) |

TABLE 2
CDRs of hu20B1 variants

| version | | | | |
|---|---|---|---|---|
| number | | CDR1 | CDR2 | CDR3 |
| v1.0 | HC | GYIFTIAGIQ | WINTHSGVPEYAEEFKG | IYYGNNGGVMDY |
| | | (SEQ ID NO: 9) | (SEQ ID NO: 10) | (SEQ ID NO: 11) |
| | LC | RASQEISDYLT | VASSLDS | LQYANYPWT |
| | | (SEQ ID NO: 12) | (SEQ ID NO: 13) | (SEQ ID NO: 14) |
| v1.1 | HC | GYIFTIAGIQ | WINTHSGVPEYAEEFKG | IYYGNNGGVMDY |
| | | (SEQ ID NO: 9) | (SEQ ID NO: 10) | (SEQ ID NO: 11) |
| | LC | RASQEISDYLT | VASSLDS | LQYANYPWT |
| | | (SEQ ID NO: 12) | (SEQ ID NO: 13) | (SEQ ID NO: 14) |
| v1.4 | HC | GYIFTIAGIQ | WINTHSGVPEYAEEFKG | IYYGNNGGVMDY |
| | | (SEQ ID NO: 9) | (SEQ ID NO: 10) | (SEQ ID NO: 11) |
| | LC | RASQEISDYLT | VASSLDS | LQYANYPWT |
| | | (SEQ ID NO: 12) | (SEQ ID NO: 13) | (SEQ ID NO: 14) |
| v1.6 | HC | GYIFTIAGIQ | WINTHSGVPEYAEEFKG | IYYGNNGGVMDY |
| | | (SEQ ID NO: 9) | (SEQ ID NO: 10) | (SEQ ID NO: 11) |
| | LC | | | |
| v1.10 | HC | GYIFTIAGIQ | WINTHSGVPEYAEEFKG | IYYGNNGGVMDY |
| | | (SEQ ID NO: 9) | (SEQ ID NO: 10) | (SEQ ID NO: 11) |
| | LC | | | |

### Example 1

Generating Mouse Anti-Staphylococcal Enterotoxin B Antibody 20B1: Generation of mouse monoclonal anti-Staphylococcal enterotoxin B (SEB) antibodies are described in PCT/US2012/57186 filed on Sep. 27, 2012, which is herein incorporated by reference in its entirety.

Murine hybridoma producing monoclonal antibody 20B1 (isotype mIgG1/) was generated from SEB-immunized BALB/c mice in the Hybridoma Facility of Albert Einstein College of Medicine (AECOM), as described in Varshney et al. JBC 286, 9737-9747 (2011), which is herein incorporated by reference in its entirety. Antibody variable (V) regions were cloned from hybridoma cultures and sequenced at AECOM. An independent V-region isolation was carried out, and identical amino acid sequences of 20B1 V-regions were obtained.

The nucleotide sequence of the mouse 20B1 heavy chain variable region (VH) is shown below (SEQ ID NO: 1, CDRs underlined):

(SEQ ID NO: 1)
CAGATCCAGTTGGTGCAGTCTGGACCTGAGCTGAAGAAGCCTGGAGAGAC
AGTCAGGATCTCCTGCAAGGCCTCTGGGTATATCTTCACAATTGCGGGAA
TACAGTGGGTGCAAAAGATGCCAGGAAGGGGTTTGAGGTGGATTGGATGG
ATAAACACCCATTCTGGAGTGCCAGAATATGCAGAAGAGTTCAAGGGACG
GTTTGCCTTTTCTTTGGAAACCTCTGCCAGAACTGCATATTTACAGATAA
GCAACCTCAAGGATGAGGACACGGCTACGTATTTCTGTGCGCGGATCTAC
TATGGTAACAACGGGGGTGTTATGGACTATTGGGGTCAAGGAACCTCAGT
CACCGTCTCCTCA

The amino acid sequence of the 20B1 VH is shown below (SEQ ID NO: 2):

(SEQ ID NO: 2, CDRs underlined)
QIQLVQSGPELKKPGETVRISCKASGYIFTIAGIQWVQKMPGRGLRWIGW
INTHSGVPEYAEEFKGRFAFSLETSARTAYLQISNLKDEDTATYFCARIY
YGNNGGVMDYWGQGTSVTVSS

The nucleotide sequence of the mouse 20B1 light chain variable region (VL) is shown below (SEQ ID NO: 3, CDRs underlined):

(SEQ ID NO: 3)
GACATCCAGATGACCCAGTCTCCATCCTCCTTATCTGCCTCTCTGGGAGA
AAGAGTCAGTCTCACTTGTCGGGCAAGTCAGGAAATTAGTGATTACTTAA
CCTGGCTTCAGCAGAAACCAGATGGAACTATTAAACGCCTGATCTACGTC
GCATCCAGTTTAGATTCTGGTGTCCCAAAAAGGTTCAGTGGCAGTAGGTC
TGGGTCAGATTATTCTCTCACCATCAGCAGCCTTGAGTCTGAAGATTTTG
CAGACTATTACTGTCTACAATATGCTAATTATCCGTGGACGTTCGGTGGA
GGCACCAAGCTGGAAATCAGA.

The amino acid sequence of the murine 20B1 VL is shown below (SEQ ID NO: 4, CDRs underlined):

(SEQ ID NO: 4)
DIQMTQSPSSLSASLGERVSLTCRASQEISDYLTWLQQKPDGTIKRLIYV
ASSLDSGVPKRFSGSRSGSDYSLTISSLESEDFADYYCLQYANYPWTFGG
GTKLEIR

For production of mouse 20B1, hybridoma cells were grown in DMEM supplemented with 5% FBS (pre-cleared on protein A to remove bovine IgG), 10% NCTC-135 medium (Life Technologies) and 1% non-essential amino acids. On day 10 of the production run, conditioned medium (CM) was centrifuged and sterile filtered through Corning 0.22 um filters. The antibody productivity was greater than 50 mg/L.

The antibody from 7.5 liters CM was bound to 122 ml MaB Select SuRe Protein A column equilibrated with PBS-CMF (137 mM NaCl, 2.7 mM KCl, 8.1 mM Na2HPO4, 2.7 mM KH2PO4, pH 7.2). The resin was washed with 10 column volumes of PBS-CMF, pH 7.2 before the antibody was eluted (0-100% step) with Protein A Elution Buffer (20 mM citric acid, 150 mM NaCl, pH 2.5). Peak fractions were neutralized to pH 7.0 with 1M Tris pH 8.0, pooled and filtered. The material was then loaded onto 320 ml Superdex 200 size-exclusion column equilibrated in PBS-CMF pH 7.2. Peak fractions were pooled and filtered. Total mouse 20B1 yield was ˜400 mg.

### Example 2

Construction of Chimeric 20B1 Antibodies: To generate chimeric 20B1 antibody heavy chain, the nucleotide sequence of 20B1 VH region (SEQ ID NO: 1) was joined to the sequence encoding human IgG1 heavy chain constant region carrying mutations L234A, L235A and G237A (hIgG1_{—}3 M also referred to herein as “TM” for triple mutant) in a proprietary expression vector. The resulting nucleotide sequence of chimeric 20B1 (20B1_chi) heavy chain is set forth in SEQ ID NO: 5, and the corresponding amino acid sequence is set forth in SEQ ID NO: 6.

To generate chimeric 20B1 antibody light chain, the nucleotide sequence of 20B1 VL region (SEQ ID NO: 3) was joined to the sequence encoding human Kappa light chain constant region in expression vector pSMEN3. In addition, two amino acid changes were introduced into mouse VL J-segment—Leu104Val and Arg107Lys—to make it identical to human JK4 sequence. The resulting nucleotide sequence of chimeric light chain is set forth in SEQ ID NO: 7, and the corresponding amino acid sequence is set forth in SEQ ID NO: 8.

The nucleotide sequence of the chimeric 20B1 antibody full-length heavy chain (SEQ ID NO: 5) is shown below (variable region in capital letters, human constant region in lower case, CDRs underlined):

(SEQ ID NO: 5)
CAGATCCAGTTGGTGCAGTCTGGACCTGAGCTGAAGAAGCCTGGAGAGAC
AGTCAGGATCTCCTGCAAGGCCTCTGGGTATATCTTCACAATTGCGGGAA
TACAGTGGGTGCAAAAGATGCCAGGAAGGGGTTTGAGGTGGATTGGATGG
ATAAACACCCATTCTGGAGTGCCAGAATATGCAGAAGAGTTCAAGGGACG
GTTTGCCTTTTCTTTGGAAACCTCTGCCAGAACTGCATATTTACAGATAA
GCAACCTCAAGGATGAGGACACGGCTACGTATTTCTGTGCGCGGATCTAC
TATGGTAACAACGGGGGTGTTATGGACTATTGGGGTCAAGGAACCTCAGT
CACCGTCTCCTCAgcgtcgaccaagggcccatcggtcttccccctggcac
cctcctccaagagcacctctgggggcacagcggccctgggctgcctggtc
aaggactacttccccgaaccggtgacggtgtcgtggaactcaggcgccct
gaccagcggcgtgcacaccttcccggctgtcctacagtcctcaggactct
actccctcagcagcgtggtgaccgtgccctccagcagcttgggcacccag
acctacatctgcaacgtgaatcacaagcccagcaacaccaaggtggacaa
gaaagttgagcccaaatcttgtgacaaaactcacacatgcccaccgtgcc
cagcacctgaagccgctggggcaccgtcagtcttcctcttccccccaaaa
cccaaggacaccctcatgatctcccggacccctgaggtcacatgcgtggt
ggtggacgtgagccacgaagaccctgaggtcaagttcaactggtacgtgg
acggcgtggaggtgcataatgccaagacaaagccgcgggaggagcagtac
aacagcacgtaccgtgtggtcagcgtcctcaccgtcctgcaccaggactg
gctgaatggcaaggagtacaagtgcaaggtctccaacaaagccctcccag
cccccatcgagaaaaccatctccaaagccaaagggcagccccgagaacca
caggtgtacaccctgcccccatcccgggaggagatgaccaagaaccaggt
cagcctgacctgcctggtcaaaggcttctatcccagcgacatcgccgtgg
agtgggagagcaatgggcagccggagaacaactacaagaccacgcctccc
gtgctggactccgacggctccttcttcctctatagcaagctcaccgtgga
caagagcaggtggcagcaggggaacgtcttctcatgctccgtgatgcatg
aggctctgcacaaccactacacgcagaagagcctctccctgtccccgggt
aaa

The amino acid sequence of the chimeric 20B1 antibody full-length heavy chain (SEQ ID NO: 6) is shown below (variable region in upper case, human constant region in lower case, CDRs underlined):

(SEQ ID NO: 6)
QIQLVQSGPELKKPGETVRISCKASGYIFTIAGIQWVQKMPGRGLRWIGW
INTHSGVPEYAEEFKGRFAFSLETSARTAYLQISNLKDEDTATYFCARIY
YGNNGGVMDYWGQGTSVTVSSastkgpsvfplapsskstsggtaalgclv
kdyfpepvtvswnsgaltsgvhtfpavlqssglyslssvvtvpssslgtq
tyicnvnhkpsntkvdkkvepkscdkthtcppcpapeaagapsvflfppk
pkdtlmisrtpevtcvvvdvshedpevkfnwyvdgvevhnaktkpreeqy
nstyrvvsvltvlhqdwlngkeykckvsnkalpapiektiskakgqprep
qvytlppsreemtknqvsltclvkgfypsdiavewesngqpennykttpp
vldsdgsfflyskltvdksrwqqgnvfscsvmhealhnhytqkslslspg
k

The nucleotide sequence of the chimeric 20B1 antibody full-length light chain (SEQ ID NO: 7) is shown below (variable region in uppercase, human constant region in lowercase, CDRs underlined):

(SEQ ID NO: 7)
GACATCCAGATGACCCAGTCTCCATCCTCCTTATCTGCCTCTCTGGGAGA
AAGAGTCAGTCTCACTTGTCGGGCAAGTCAGGAAATTAGTGATTACTTAA
CCTGGCTTCAGCAGAAACCAGATGGAACTATTAAACGCCTGATCTACGTC
GCATCCAGTTTAGATTCTGGTGTCCCAAAAAGGTTCAGTGGCAGTAGGTC
TGGGTCAGATTATTCTCTCACCATCAGCAGCCTTGAGTCTGAAGATTTTG
CAGACTATTACTGTCTACAATATGCTAATTATCCGTGGACGTTCGGTGGA
GGCACCAAGGTGGAAATCAAAcgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt

The amino acid sequence of the chimeric 20B1 antibody full-length light chain (SEQ ID NO: 8) is shown below (variable region in uppercase, human constant region in lowercase, CDRs underlined):

(SEQ ID NO: 8)
DIQMTQSPSSLSASLGERVSLTCRASQEISDYLTWLQQKPDGTIKRLIYV
ASSLDSGVPKRFSGSRSGSDYSLTISSLESEDFADYYCLQYANYPWTFGG
GTKVEIKrtvaapsvfifppsdeqlksgtasvvcllnnfypreakvqwkv
dnalqsgnsqesvteqdskdstyslsstltlskadyekhkvyacevthqg
lsspvtksfnrgec

### Example 3

Humanization of Mouse 20B1 Antibody: The CDRs of the mouse antibody 20B1 VH were identified using the AbM definition, which is based on sequence variability as well as the location of the structural loop regions. Kabat definition was used for VL CDRs. The sequences of 20B1 heavy chain CDRs are shown in SEQ ID NO:9 (CDR-H1), SEQ ID NO:10 (CDR-H2) and SEQ ID NO:11 (CDR-H3), and the 20B1 light chain CDRs are shown in SEQ ID NO: 12 (CRD-L1), SEQ ID NO:13 (CDR-L2) and SEQ ID NO:14 (CDR-L3). See Table 2.

For humanization of the heavy chain, VH CDRs were first grafted onto human germline acceptor framework DP-54 (GenBank accession No. CAA78224.1). For the J-segment, human IGHJ4 sequence was used. The resulting amino acid sequence of hu20B1 VH v1.0, is shown in SEQ ID NO: 15. See Table 1. The corresponding nucleic acid sequence is shown in SEQ ID NO: 17.

hu20B1 VH1.0—Nucleic Acid (CDR-Encoding Regions Underlined):

(SEQ ID NO: 17)
gaggtgcagctggtggagtctgggggaggcttggtccagcctggggggtc
cctgagactctcctgtgcagcctctgggtatatcttcacaattgcgggaa
tacagtgggtccgccaggctccagggaaggggctggagtgggtggcctgg
ataaacacccattctggagtgccagaatatgcagaagagttcaagggacg
attcaccatctccagagacaacgccaagaactcactgtatctgcaaatga
acagcctgagagccgaggacacggctgtgtattactgtgcgagaatctac
tatggtaacaacgggggtgttatggactattggggccaaggaaccctggt
caccgtctcctca

For humanization of the light chain, VL CDRs were first grafted onto human DPK9 germline acceptor framework (GenBank accession No. X93627.1), which shows high degree of similarity to 20B1 VL. For the J-segment, human IGKJ4 sequence was used. The amino acid sequence, hu20B1 VL v1.0, is set forth in SEQ ID NO: 16. See Table 1. The hu20B1 VL v1.0 is encoded by the nucleic acid sequence shown in SEQ ID NO: 20.

hu20B1 VL1.0—Nucleic Acid (CDR-Encoding Regions Underlined):

(SEQ ID NO: 20)
gacatccagatgacccagtctccatcctccctgtctgcatctgtaggaga
cagagtcaccatcacttgccgggcaagtcaggaaattagtgattacttaa
cctggtatcagcagaaaccagggaaagcccctaagctcctgatctatgtc
gcatccagtttagattctggggtcccatcaaggttcagtggcagtggatc
tgggacagatttcactctcaccatcagcagtctgcaacctgaagattttg
caacttactactgtctacaatatgctaattatccgtggacgttcggcgga
gggaccaaggtggagatcaaa.

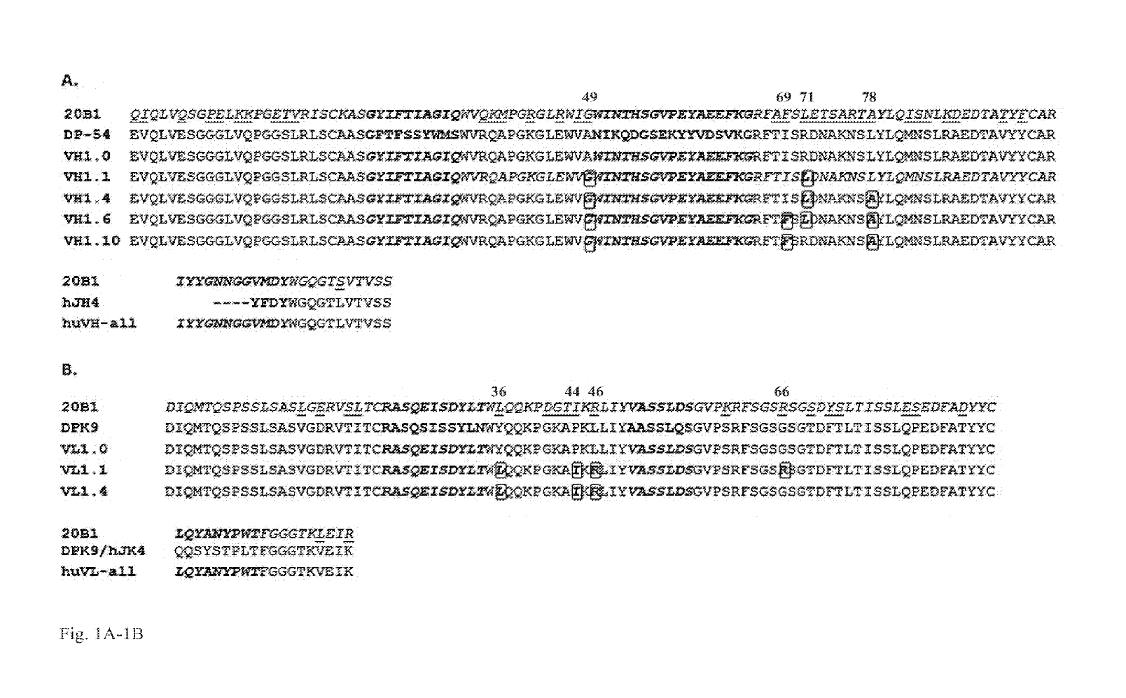

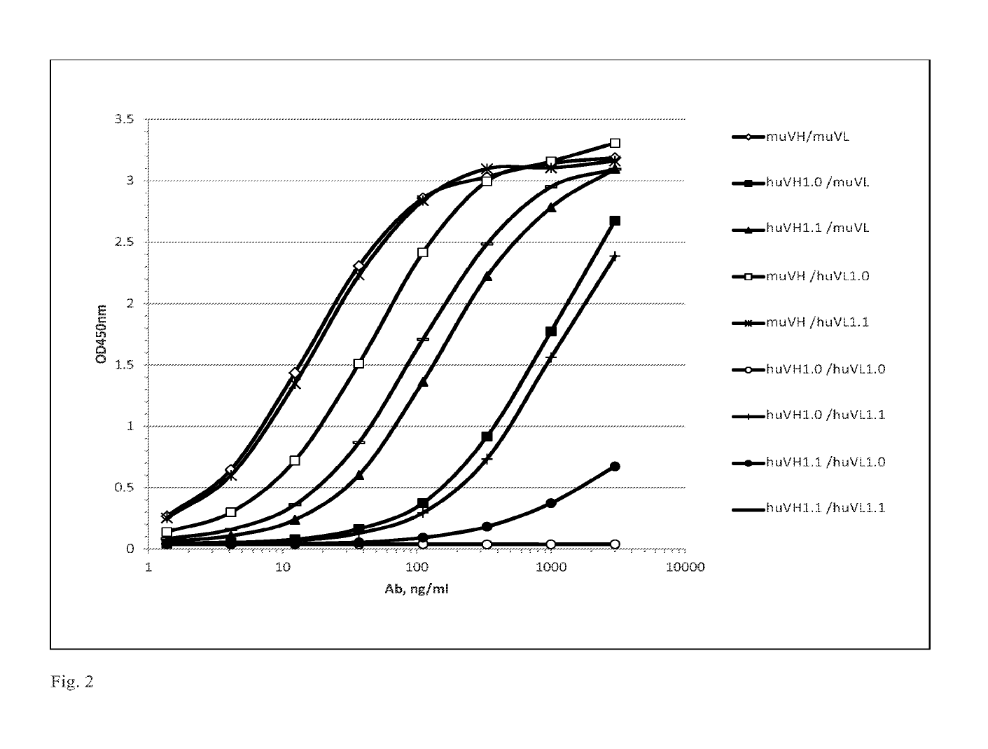

Testing CDR-grafted v1.0 variants of humanized 20B1 showed that most of the SEB binding activity was lost (see Example 5 below). Thus, additional humanized variants were constructed, where one or several back-mutations (i.e., residues corresponding to murine 20B1 sequence) were introduced into human DP-54 and DPK9 framework sequences. For humanized 20B1 VH v1.1 (SEQ ID NO: 19 (amino acid) and SEQ ID NO: 31 (nucleic acid)), back-mutations Ala49Gly and Arg71Leu (Kabat numbering) were introduced (Table 4 and FIG. 1).

hu20B1 VH1.1 Nucleic Acid (CDR-Encoding Regions Underlined):

(SEQ ID NO: 31)
gaggtgcagctggtggagtctgggggaggcttggtccagcctggggggtc
cctgagactctcctgtgcagcctctgggtatatcttcacaattgcgggaa
tacagtgggtccgccaggctccagggaaggggctggagtgggtgggatgg
ataaacacccattctggagtgccagaatatgcagaagagttcaagggacg
attcaccatctccctggacaacgccaagaactcactgtatctgcaaatga
acagcctgagagccgaggacacggctgtgtattactgtgcgagaatctac
tatggtaacaacgggggtgttatggactattggggccaaggaaccctggt
caccgtctcctca.

For humanized 20B1 VH v1.4 (SEQ ID NO: 21 and SEQ ID NO: 22), back-mutations Ala49Gly, Arg71Leu and Leu78Ala were made. Likewise, 20B1 VH versions 1.6 and 1.10 were generated. Back-mutations were also introduced into 20B1 VL1.0 to generate variants VL1.1 and VL1.4 (Table 4 and FIG. 1).

TABLE 4
Humanized variants of 20B1 heavy and
light chain with back-mutations.

| | Back-mutations in | |
|---|---|---|
| Humanized 20B1 V_{H} | human frameworks | Amino acid |
| or V_{L} variant | (Kabat numbering) | sequence |
| hu20B1 V_{H} 1.1 | A49G, R71L | SEQ ID NO: 19 |
| hu20B1 V_{H} 1.4 | A49G, R71L, L78A | SEQ ID NO: 21 |
| hu20B1 V_{H} 1.6 | A49G, I69F, R71L, L78A | SEQ ID NO: 23 |
| hu20B1 V_{H} 1.10 | A49G, I69F, L78A | SEQ ID NO: 25 |
| hu20B1 V_{L} 1.1 | Y36L, P44I, L46R, G66R | SEQ ID NO: 27 |
| hu20B1 V_{L} 1.4 | Y36L, P44I, L46R | SEQ ID NO: 29 |

### Example 4

Expression and Purification of Chimeric and Humanized 20B1 antibodies: For small-scale antibody generation and testing, chimeric and humanized 20B1 anti-SEB antibodies were expressed in COS-1 cells by transient transfection with a combination of desired heavy and light chain DNA constructs in proprietary vectors. Antibodies in CM collected 3 days post-transfection were quantified using hIgG1-specific ELISA and used for binding studies following spin-column purification on protein A or without purification.

For a large-scale expression, antibodies were transiently expressed in HEK293F cells and purified from 5-day CM using protein A affinity and size exclusion chromatography, as described below.

Cell Culture and Transfections: HEK293F cells (American Type Culture Collection) were cultured in FreeStyle™293 expression medium (Life Technologies). Cells were grown and maintained in a humidified incubator with 7% CO2 at 37° C. Conditioned media were produced from a large-scale transient HEK293 transfection process. Specifically, per 1 liter of suspension HEK293 cells were transfected using 0.5 mg of each heavy and light chain plasmid DNA constructs according to standard methods known in the art. Transfected cells were grown in wave bioreactors incubated at 37° C. with a rocking rate of 20 rpm for 120 hrs before harvest. The conditioned media were filtered at 0.22 m prior to purification.

Purification of 20B1 antibodies expressed in HEK293F cells: 5 liters of unconcentrated CM was filtered through a 0.2 um PES filter. The antibody was bound to 65 ml MaB Select SuRe Protein A column equilibrated with PBS-CMF, pH 7.2. The resin was washed with 10 CVs of PBS-CMF pH 7.2 before the antibody was eluted (0-100% step) with Protein A Elution Buffer (20 mM citric acid, 150 mM NaCl, pH 2.5). Peak fractions were neutralized to pH 7.0 with 1M Tris pH 8.0 and were pooled and filtered. The material was loaded onto 320 ml Superdex 200 size-exclusion column equilibrated in PBS-CMF pH 7.2. Peak fractions were pooled and filtered. The pool was concentrated using 30 kDa spin filters and frozen.

### Example 5

Determining Antibody Binding Affinity: This example illustrates the determination of antibody binding affinity for Staphylococcal enterotoxin B antibodies. Binding of hu20B1 variants to SEB was evaluated using the following techniques: 1) Direct ELISA on SEB-coated plate, 2) Competition ELISA of unlabeled antibodies with biotinylated reference antibody (chimeric 20B1), and 3) ForteBio's Octet® technology all according to standard methods known in the art.

Direct SEB ELISA: SEB protein (obtained from Toxin Technologies Inc., cat# BT202) was coated on Costar high-binding 96-well ELISA plates at 1 g/ml in PBS overnight at 4° C. The plates were blocked for 3 hrs at room temperature with 0.5% BSA in PBS+0.05% Tween-20 (assay buffer). Diluted CM or purified 20B1 antibodies diluted in assay buffer were added for lhr, followed by goat anti-hFc-HRP (Thermo) for lhr. TMB substrate solution was added and absorbance at 450 nm was measured after stopping the reaction with 0.18M H2504. The data were analyzed with Microsoft Excel and GraphPad Prism software.

FIG. 2 shows a direct ELISA binding experiment with chimeric and hu20B1 antibodies, including “semi-humanized” variants, where a humanized heavy or light chain is combined with a chimeric light or heavy chain. This experiment demonstrates significant loss of binding activity for CDR-grafted (v1.0) versions of heavy and light chains of 20B1, but partial restoration of binding upon introduction of back-mutations, i.e., in versions VH v1.1 and VL v1.1. The data also demonstrate that additional back-mutations are needed, especially for the VH, to fully restore SEB binding. Therefore, more back-mutations were explored as outlined in Example 3 above.

Competition ELISA: Chimeric 20B1-hIgG1_{—}3 M protein (ch20B1) was biotinylated in PBS buffer using amino-reactive Sulfo-NHS-LC-biotin reagent (Thermo) for 2 hrs on ice, at 20:1 reagent:antibody molar ratio, then dialyzed extensively against PBS. Binding of biotin-ch20B1 to SEB was first measured by direct SEB ELISA as described above, except using streptavidin-HRP (StrAv-HRP) for detection. From this experiment, a concentration of 50 ng/ml biotin-ch20B1 (in the near-saturation range of the binding curve) was chosen for competition ELISA.

In competition ELISA, plates were coated with SEB protein and blocked as described for direct ELISA. Then, a mixture of biotin-ch20B1 at 50 ng/ml and unlabeled competitor Ab (at varying concentrations) was added and incubated for lhr. Bound biotin-ch20B1 was detected using StrAv-HRP.

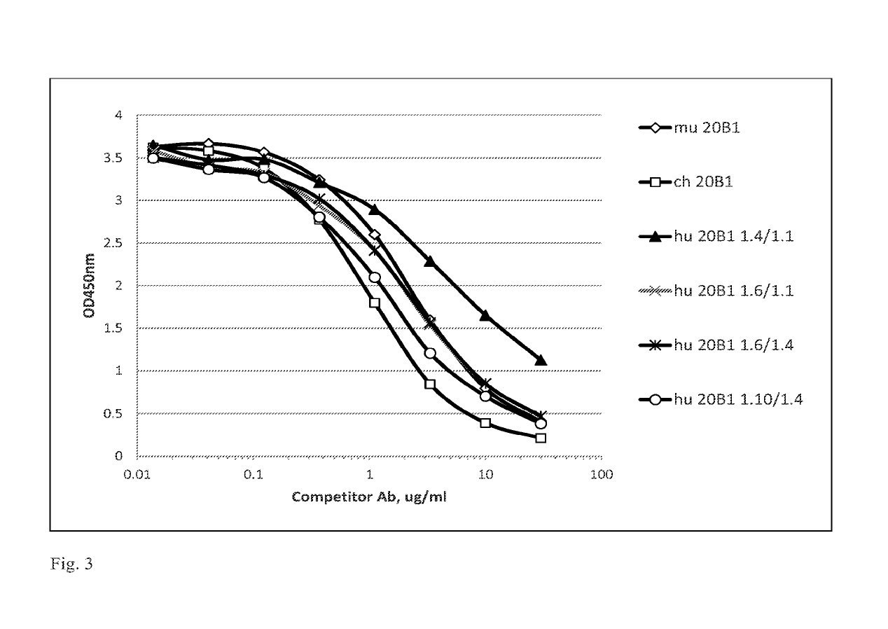

FIG. 3 shows a graph showing the results of competition ELISA experiment with murine, chimeric and four humanized variants of mAb 20B1. The data demonstrate that good restoration of SEB binding in hu20B1 variants 1.6/1.1, 1.10/1.1 and 1.10/1.4, and somewhat less in 1.4/1.1, was achieved.

To obtain quantitative kinetic values of binding of murine and humanized 20B1 antibodies to SEB, a series of Octet experiments was performed. Importantly, Octet measures the binding of immobilized antibodies to SEB present in solution, which may be more relevant to the mechanism of antibody-mediated neutralization of SEB in vivo. Octet

SEB binding experiments were carried out using Octet QKe system and analyzed with Octet v7.0 software (ForteBio). Antibody 20B1 variants were captured at 10 ug/ml on anti-mIgG Fc or anti-hIgG Fc kinetic sensors in 80 ul of 1× Kinetic Buffer (ForteBio). An unrelated reference antibody (mIgG1 or hIgG1) was used to measure and subtract any non-specific binding of SEB to antibody-coated biosensor; very little, if any, such binding was detected at all tested SEB concentrations. Captured antibodies were exposed to SEB at several concentrations, typically starting at 25 nM with several 2-fold dilutions. A sensor exposed to buffer alone (no SEB) was used as a second reference for signal subtraction. Typical assay steps included the following: sensor check (15s), Ab loading (600s), baseline (180s), association (600s), and dissociation (1800-2700s). Binding curves obtained after double-reference subtractions were analyzed to obtain the kinetic parameters (Ka, Kd and KD).

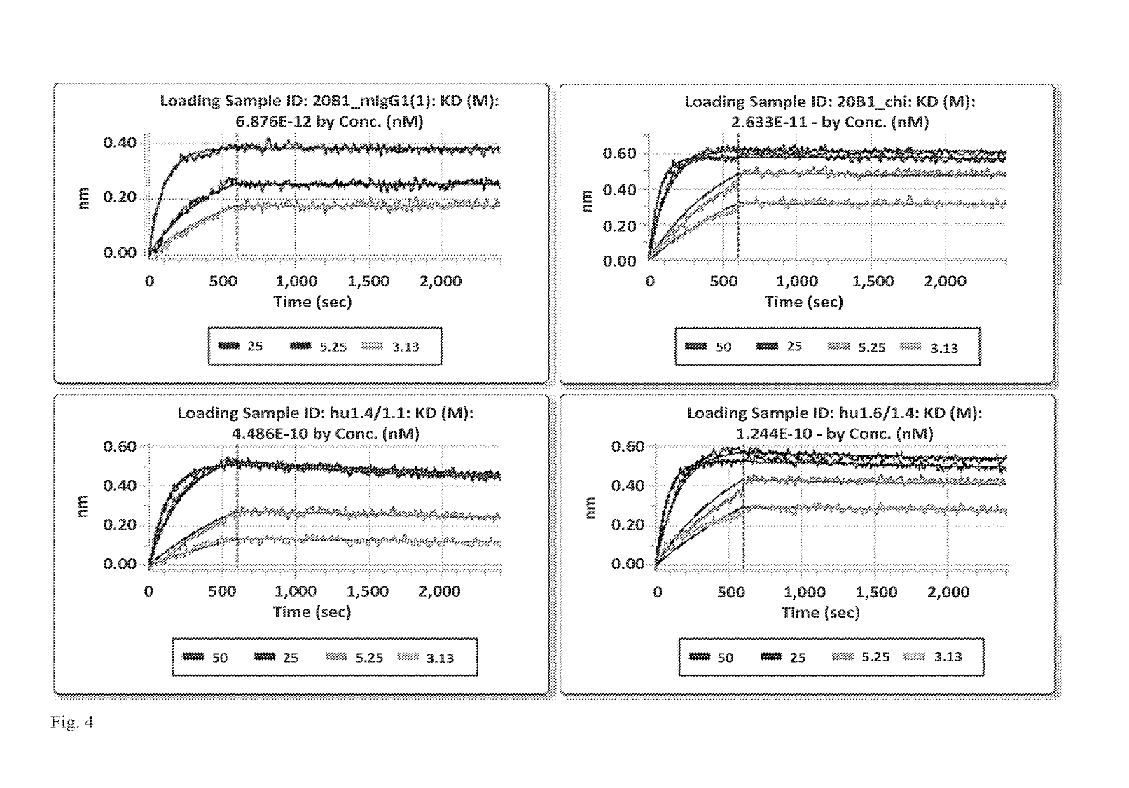

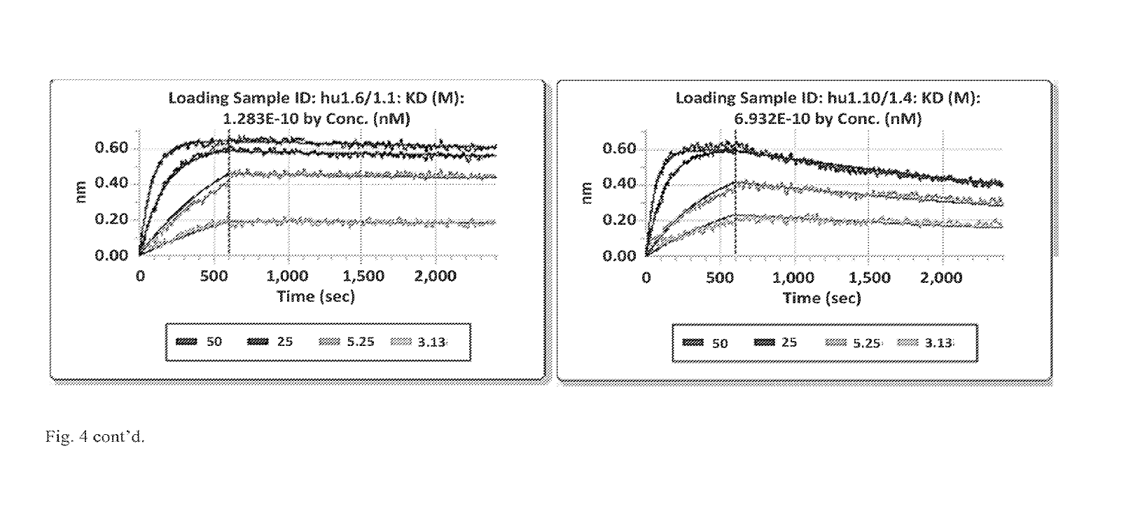

FIG. 4 shows examples of Octet binding curves for mu20B1, ch20B1 and hu20B1 v1.4/1.1, v1.6/1.1, v1.6/1.4 and v1.10/1.4 antibodies. The kinetic parameters of SEB-antibody interaction are summarized in Table 5. This experiment demonstrates that hu20B1 v1.6/1.1 and v1.6/1.4 both have binding affinity to SEB within ˜2-fold of the parental mouse 20B1. Surprisingly, variant 1.10/1.4 has affinity reduced ˜14-fold, in contrast to competition ELISA result in FIG. 3. The variant 1.4/1.1 demonstrated an intermediate degree of reduction of binding, ˜7-fold.

TABLE 5
Kinetic Parameters of Antibody Binding to SEB Obtained By Octet.
Data for Duplicate Experiments are shown

| Antibody | KD (M) | Ka (1/Ms) | Ka error | Kd (1/s) | Kd error | KD, average (pM) |
|---|---|---|---|---|---|---|
| mu20B1 | 7.86E−11 | 3.37E+05 | 5.96E+03 | 2.65E−05 | 1.39E−06 | 62.0 |
| | 4.53E−11 | 4.35E+05 | 8.83E+03 | 1.97E−05 | 1.57E−06 | |
| ch20B1 | 2.63E−11 | 2.99E+05 | 4.43E+03 | 7.88E−06 | 2.07E−06 | 23.0 |
| | 1.90E−11 | 2.82E+05 | 3.40E+03 | 5.35E−06 | 1.71E−06 | |
| hu20B1 | 4.49E−10 | 1.67E+05 | 2.25E+03 | 7.50E−05 | 2.14E−06 | 442.0 |
| v1.4/1.1 | 4.35E−10 | 1.13E+05 | 2.13E+03 | 4.93E−05 | 2.61E−06 | |
| hu20B1 | 1.28E−10 | 2.35E+05 | 2.87E+03 | 3.01E−05 | 1.82E−06 | 129.0 |
| v1.6/1.1 | 1.30E−10 | 2.51E+05 | 2.17E+03 | 3.25E−05 | 1.28E−06 | |
| hu20B1 | 1.24E−10 | 2.59E+05 | 4.32E+03 | 3.23E−05 | 2.41E−06 | 112.0 |
| v1.6/1.4 | 9.98E−11 | 2.59E+05 | 2.71E+03 | 2.58E−05 | 1.54E−06 | |
| hu20B1 | 6.93E−10 | 3.12E+05 | 4.76E+03 | 2.16E−04 | 2.51E−06 | 882.0 |
| v1.10/1.4 | 1.07E−09 | 2.61E+05 | 4.67E+03 | 2.79E−04 | 3.13E−06 | |

### Example 6

Staphylococcal Enterotoxin B Antibodies Neutralize the Mitogenic Effect of Staphylococcal Enterotoxin B on murine cells: This example illustrates the effect of Staphylococcal enterotoxin B antibodies in a mouse splenocyte cell based assay. The antibodies were tested in the murine cell based proliferation assay to generate cellular IC50 and confirm inhibitory effect of SEB mitogenic activity.

RPMI 1640 with L-glutamine, 10% FBS with 1% Penicillin-Streptomycin antibiotics added was obtained from Cellgro (Manassas, Va.). HI FBS in 500 mL bottles, obtained from Gibco (Grand Island, N.Y.), was thawed and aliquoted in 50 mL. For each 500 mL of Media needed, 5 mL of Pen-Strep and 50 mL of FBS were added.

Cells were counted using the Countess automated cell counter. Costar 3799, 96-well plates were used for cell culture, and Costar 3917, 96-well plates were used after the cells were lysed.

The antibodies that were expressed, purified and analyzed are summarized in Table 6. Antibodies were shipped frozen, thawed at room temperature and then stored at 4° C. The antibodies were diluted into media for each assay to the target concentration of 10 μg/mL.

TABLE 6
Antibodies Tested in Murine Cell Proliferation Assay. (TM =
Triple Mutant, 3 mutations - L234A/L235A/G237A - in the IgG1 heavy
chain constant region to eliminate binding to Fc gamma receptor)

| Antibody | Concentration (mg/mL) |
|---|---|
| Murine IgG1/κ 20B1 Native Protein | 6 |
| Anti Eimeria tenella muIgG 1/κ Isotype control | 4.8 |
| Chimeric 20B1 with huIgG1TM | 0.95 |
| Anti Tetanus Toxin huIgG1 TM | 7.95 |
| Isotype control | |
| hu20B1v1.4/1.1 huIgG1 TM | 2.56 |
| hu20B1v1.6/1.1 huIgG1 TM | 3 |
| hu20B1v1.6/1.4 huIgG1 TM | 2.98 |
| Hu20B1v1.10/1.4 huIgG1 TM | 2.84 |

The cell mitogen, Staphylococcus Enterotoxin B (SEB), was obtained from Toxin Technology (Sarasota, Fla.) and aliquoted at 0.1 mg/mL, and then frozen at −80° C. One aliquot was thawed at RT and used per assay. A diluted aliquot was made with a target concentration of 100 ng/mL. ViaLight plus kit LT07-221 was manufactured at Lonza and was used to measure total cell proliferation. To achieve optimal performance, Protocol 2 was used for each experiment. The Wallac EnVision machine (PerkinElmer, Waltham, Mass.) was used to read bioluminescence ATP monitoring reagent.

Murine splenocyte single cell suspension was produced by macerating freshly excised spleen from 6 to 8 week old normal female BALB/c mice from Albert Einstein College of Medicine (AECOM). The spleen was placed in 10 mL of RPMI media. The freshly excised spleen was subsequently macerated with into the media. Cell suspension was then placed in a 50 mL falcon tube and was centrifuged at 1,200 rpm for 5 min. RBC were then lysed, the solution strained, and the cells were seeded into a 96-well plate. Test antibodies were then added to wells at the indicated concentration. Nanomolar concentration is based on assumed molecular weight of approximately 145kD. SEB was then added to final concentration of 25 ng/mL. Cells were incubated at 37° C., with 5% CO2 for 4 days.

FIG. 5 shows the effect of mouse anti-SEB monoclonal antibody clone 20B1 and anti-E. tenella on SEB-induced murine cell proliferation. The circles represent an average of triplicates of the relative fluorescence units (RFU). The triangles represent an average RFU of a monoclonal antibody directed against E. tenella, a physiologically irrelevant antibody. The dotted line represents the cellular IC₅₀. Percentage of control for zero percent is zero stimulant added. Percentage of control for zero percent is zero stimulant added. Percentage of control for 100 percent is 25 ng/mL SEB. FIG. 5 demonstrates that anti-SEB monoclonal antibody 20B1 almost fully inhibits SEB-induced murine splenocyte proliferation down to a level of approximately 5%, with IC₅₀ value of 0.467 nM, which is within normal expected background signal for this assay system, relative to an isotype control antibody.

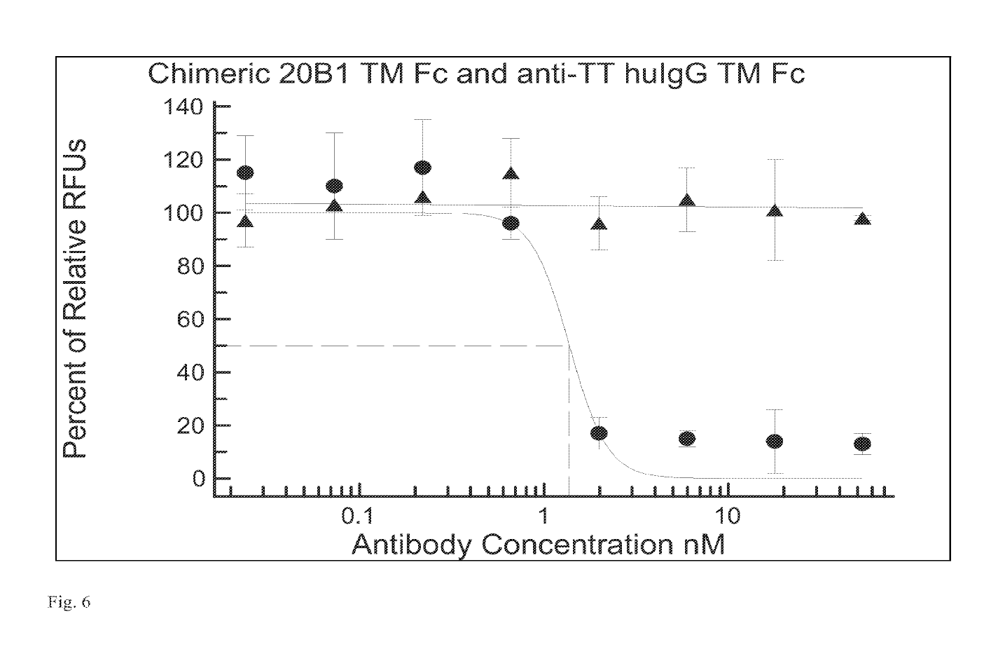

FIG. 6 shows the effect of chimeric 20B1 TM Fc and anti-TT human IgG TM Fc on SEB-induced murine cell proliferation. The circles represent an average of triplicates of the relative fluorescence units (RFU). The triangles represent an average RFU of a monoclonal antibody directed against E. tenella, a physiologically irrelevant antibody. The dotted line represents the cellular IC₅₀. Percentage of control for zero percent is zero stimulant added. Percentage of control for 100 percent is 25 ng/mL SEB. FIG. 6 demonstrates that chimeric antibody 20B1 also almost fully inhibits SEB-induced murine splenocyte proliferation down to a level of approximately 5%, with IC₅₀ value of 0.467 nM, which is within normal expected background signal for this assay system, relative to an isotype control antibody.

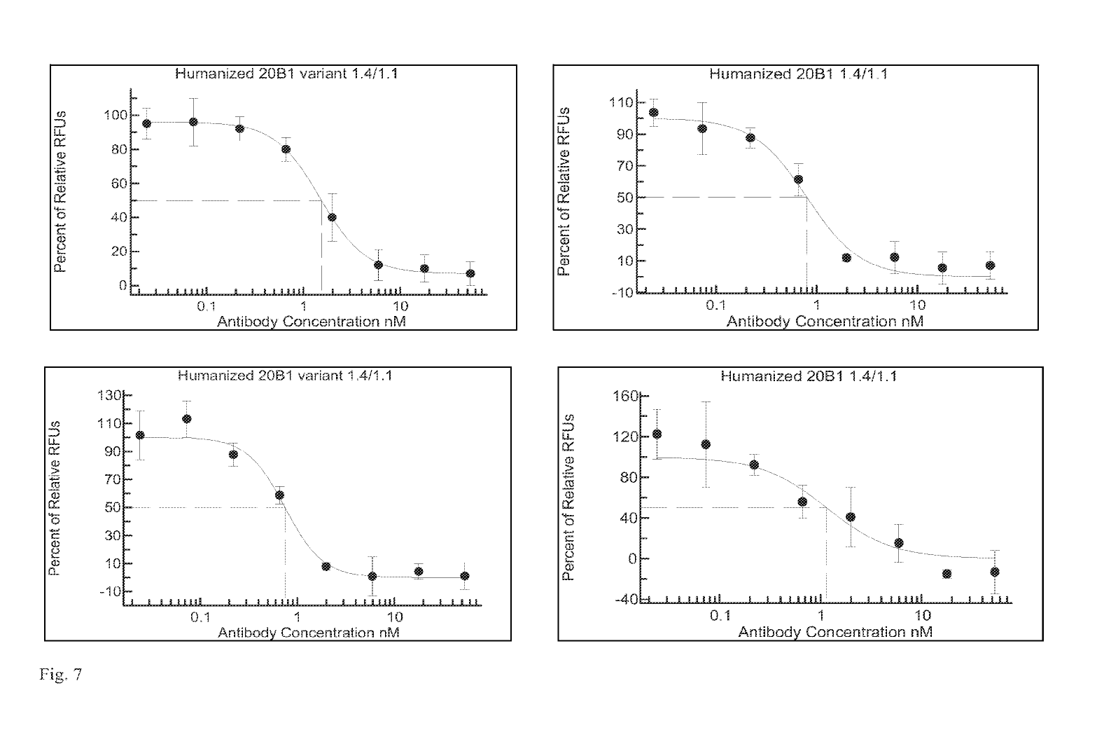

FIG. 7 shows the effect of humanized 20B1 variant 1.4/1.1 on SEB-induced murine splenocyte proliferation. The circles represent an average of triplicates of the relative fluorescence units (RFU). The dotted line represents the cellular IC₅₀. Percentage of control for zero percent is zero stimulant added. Percentage of control for 100 percent is 25 ng/mL SEB. FIG. 7 demonstrates that humanized 20B1 variant 1.4/1.1 antibody fully inhibits SEB-induced murine splenocyte proliferation, with an IC₅₀ value of 0.7-1.5 nM.

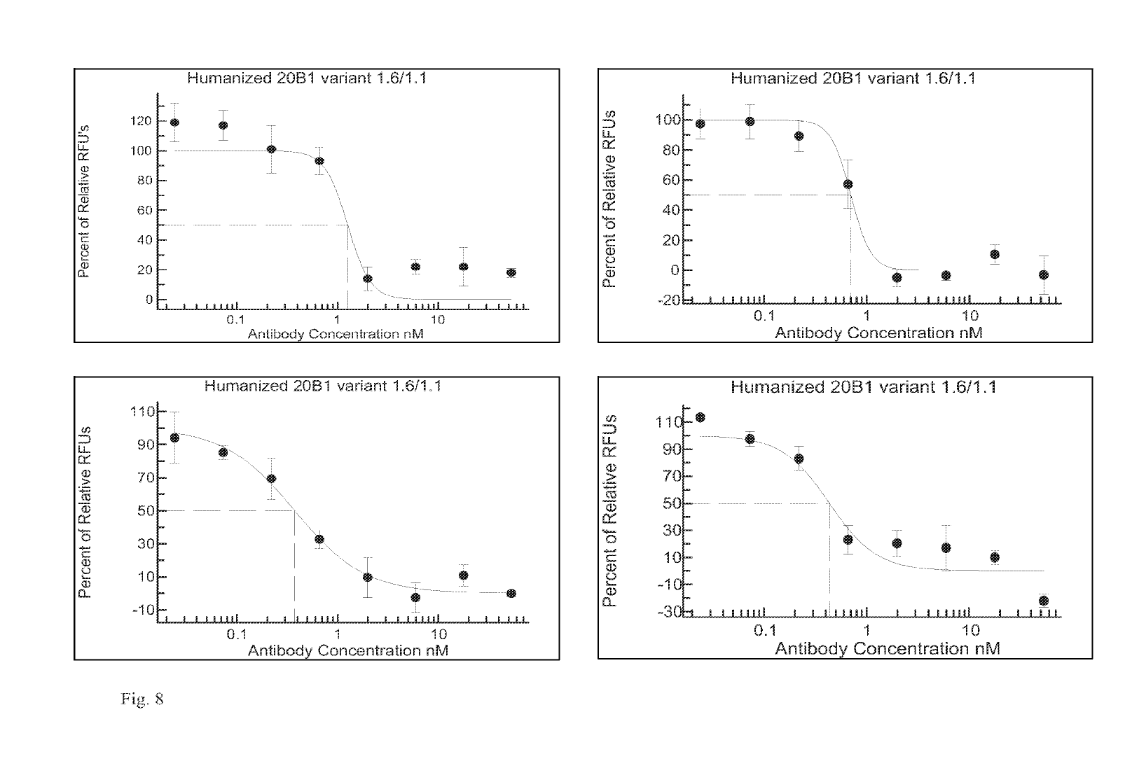

FIG. 8 shows the effect of humanized 20B1 variant 1.6/1.1 (hu20B1v1.6/1.1) on SEB-induced murine splenocyte proliferation. The circles represent an average of triplicates of the relative fluorescence units (RFU). The dotted line represents the cellular IC50. Percentage of control for zero percent is zero stimulant added. Percentage of control for 100 percent is 25 ng/mL SEB. FIG. 8 demonstrates that hu20B1v1.6/1.1 (humanized 20B1 antibody comprising VL version 1.6 and VL version 1.1) antibody fully inhibits SEB-induced murine splenocyte proliferation, with an IC₅₀ value of 0.3-1.2 nM.

These results demonstrate that Staphylococcal enterotoxin B antibodies are effective in neutralizing the mitogenic effect of Staphylococcal enterotoxin B on murine cells.

### Example 7

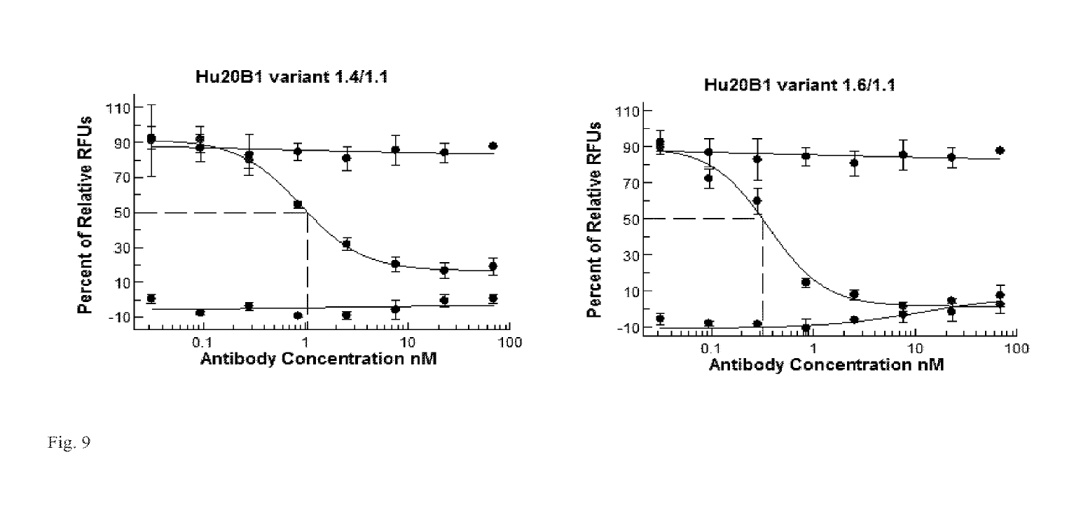

In vitro neutralization activity of hu20B1v1.4/1.1 and hu20B1v1.6/1.1: Inhibition of SEB induced T cell proliferation by humanized antibodies was also tested in a toxin neutralization assay using human PBMC. In these experiments, after incubation for 72 h, proliferation was measured in supernatants, and the percentage inhibition was calculated using XLfit software. Both hu20B1v1.4/1.1 and hu20B1v1.6/1.1 inhibited SEB induced T-cell proliferation in human PBMCs at an IC₅₀ of 1.05 nM and 0.32 nM respectively (See FIG. 9). These concentrations were comparable to murine mAb 20B1 which inhibited the T cell proliferation at an IC₅₀ 0.74 nM in human PBMCs. In contrast, isotype control mAb did not inhibit SEB induced T cell proliferation.

### Example 8

Therapeutic efficacy of Hu-mAbs in SEB induced lethal shock: The protective efficacy of hu20B1v1.4/1.1 and hu20B1v1.6/1.1 was explored in in vivo in BALB/c mice model for SEBILS. Both Humanized mAb demonstrated consistent protection and comparable to murine mAb 20B1 in the D-galactosamine potentiated mice (see FIG. 10A). IFN-γ levels in the blood of mAb or PBS treated mice 2h and 8h after the SEB intoxicaion were also invesitgated. As shown in FIG. 10B, IFN-γ levels were elevated only in PBS control compared to mAb treated mice at both time point.

### Example 9

Treatment with humanized mAb 20B1 decreases inflammatory response and T-cell proliferation: Analysis of peripheral complete white blood cell count revealed significantly lower leukocyte counts in hu20B1v1.4/1.1 and hu20B1v1.6/1.1 when compared to PBS treated mice. Leukocyte counts were comparable to those obtained in mice treated with vancomycin and not significantly elevated when compared to sham infected mice. (FIG. 11A). Neutrophil and eosinophil counts were also lower in the group treated with hu20B1v1.4/1.1 and hu20B1v1.6/1.1, murine 20B1 or vancomycin

Multiplex cytokine analysis of abscess fluid yielded significantly lower levels of mIL-1ra (FIG. 11B) in abscesses of hu20B1v1.4/1.1 and hu20B1v1.6/1.1 treated mice and a similar trend of reduction for mIL-6 These cytokine levels were not significantly reduced in vancomycin treated mice. In addition, no difference in IL-2, IL-4, IL-5, IL-10, IL-12, IFN-γ and TNF-α was observed in the abscesses of mice in any of the groups.

Next, groin lymph nodes were excised to determine size, and quantify lymphocyte count by FACS. Enlarged lymph nodes were seen in mice from the PBS treated and isotype control Ab treated group. Similar trend was seen and the number of lymphocytes was higher in PBS treated mice compared to humanized or murine mAb treated mice.

### Example 10

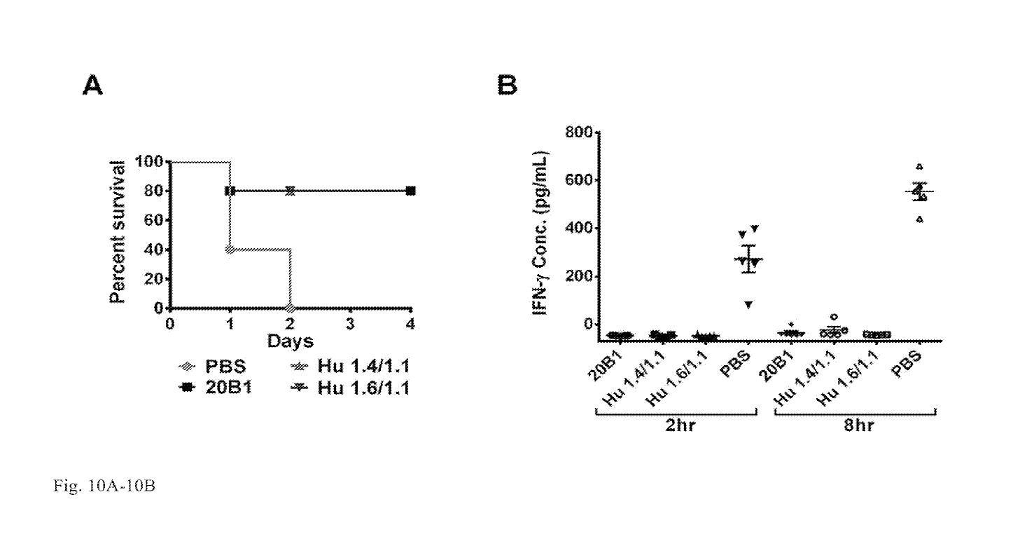

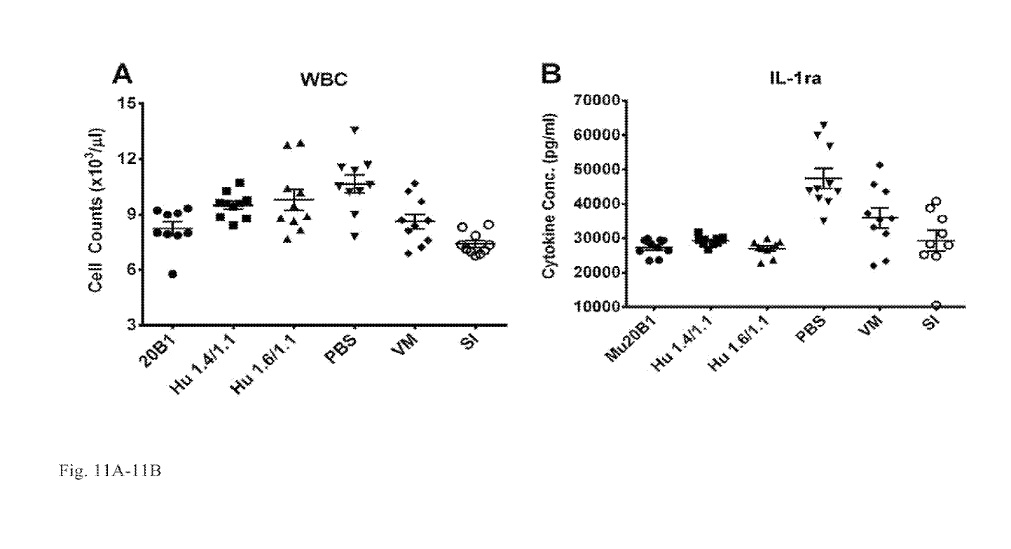

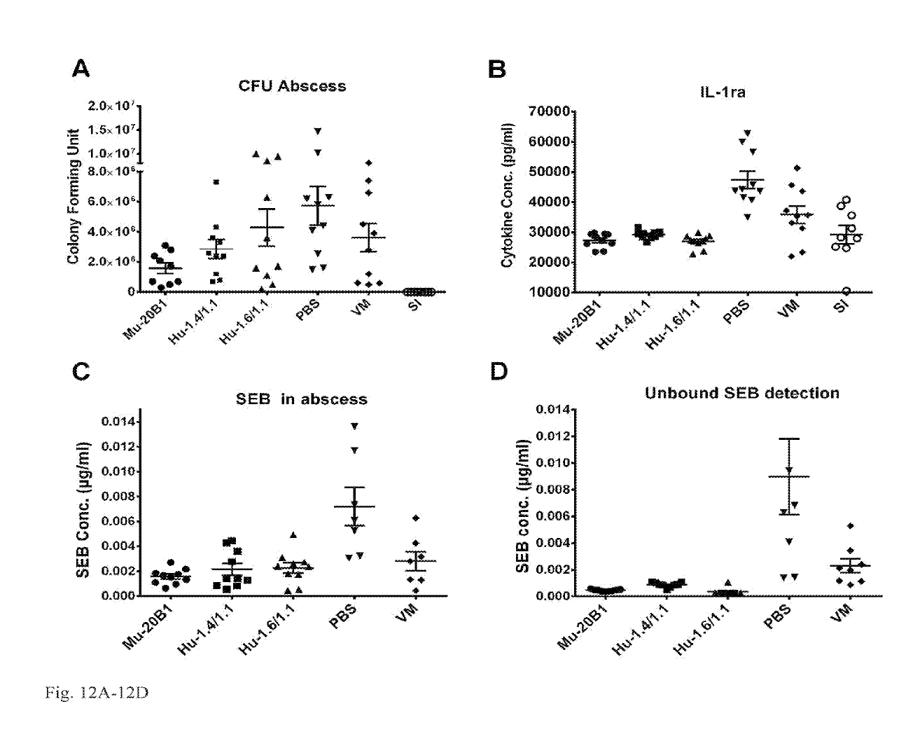

Treatment with mAb 20B1 reduces the abscess size and enhanced clearance of S. aureus and SEB: Next, the efficacy of Hu-mAbs was further explored in a murine thigh infection model, which mimics complicated deep tissue skin infection. Significant abscess size reduction was observed by day 5 in mice treated with hu20B1v1.4/1.1 and hu20B1v1.6/1.1 compared to PBS. Consistent with that finding, successful treatment with hu20B1v1.4/1.1 and hu20B1v1.6/1.1 resulted in 2 fold and 1.5 fold CFU reduction in the abscess of those mice (FIG. 12A). The infection remained localized and no dissemination of S. aureus to spleen was documented in any of the experimental groups.

The efficacy of Hu-1.4/1.1 and Hu-1.6/1.1 was explored in a murine thigh model infection. Abcess size reduction was observed in Hu-1.4/1.1 and Hu-1.6/1.1 treated mice by day 5. CFU was lower in abscess of Hu-1.4/1.1 and Hu-1.6/1.1 compared to PBS treated mice (FIG. 12A). As previously observed, the infection remained localized. Analysis of peripheral complete white blood cell count revealed a trend of lower leukocytes and neutrophil counts in the mAb treatment group compared to PBS group. Multiplex cytokine analysis of abscess fluid yielded significantly lower level of mIL-1ra (FIG. 12B) in abscesses of Hu-1.4/1.1, Hu-1.6/1.1 and murine mAb 20B1 treated mice when compared to PBS treated mice (p<0.01) whereas IL-2, IL-4, IL-5, IL-10, IL-12, IFN-γ and TNF-α levels in abscesses of mice were comparable. ELISA analysis confirmed the presence of SEB in the abscesses of all mice. Consistent with reduced abscess size and CFU, SEB levels were lower in the mAb treated mice compared to PBS, or vancomycin treated mice (FIG. 12C). Additional ELISA experiments showed significantly lower level of unbound SEB in the abscess of Hu-mAb 1.4/1.1, 1.6/1.1 and mAb 20B1 treated mice compared to PBS treated mice (p<0.05) when detection was performed with IgG2a isotype switch variants of mAb-20B1 (FIG. 12D). In contrast, SEB remained detectable in PBS, and vancomycin-treated mice. This finding was consistent with the conclusion that Hu-mAb 1.4/1.1, 1.6/1.1 bound their target SEB in vivo.

### Example 11

SEB specific mAb decreases SEB levels in abscess and is bound to mAb: Proof of mechanism was further explored by documenting that SEB specific mAbs binds the target antigen SEB in the abscess. First ELISA experiments confirmed SEB was excreted by MRSA strain 38 in vivo in the abscesses of all mice except sham-infected mice. Consistent with reduced abscess size and pathogen burden SEB levels were lower in the humanized or murine mAb treated mice compared to PBS treated mice (FIG. 12C). Additional ELISA experiments were done comparing SEB detection with IgG2a isotype switch variants of mAb 20B1 and mAb 6D3, which bind to a non-overlapping epitope on SEB. These experiments confirmed that SEB was only detected in the abscess of mAb 20B1 treated mice if mAb-6D3 (IgG2a) and not mAb-20B1 (IgG2a) was used as the detection Ab. In contrast, SEB remained detectable in PBS, and vancomycin-treated mice when mAb-20B1 (IgG2a) was employed as the detection mAb in the capture ELISA. This finding was consistent with the conclusion that SEB was bound in the abscess by humanized or murine mAb 20B1 in the treatment group.

### Example 12

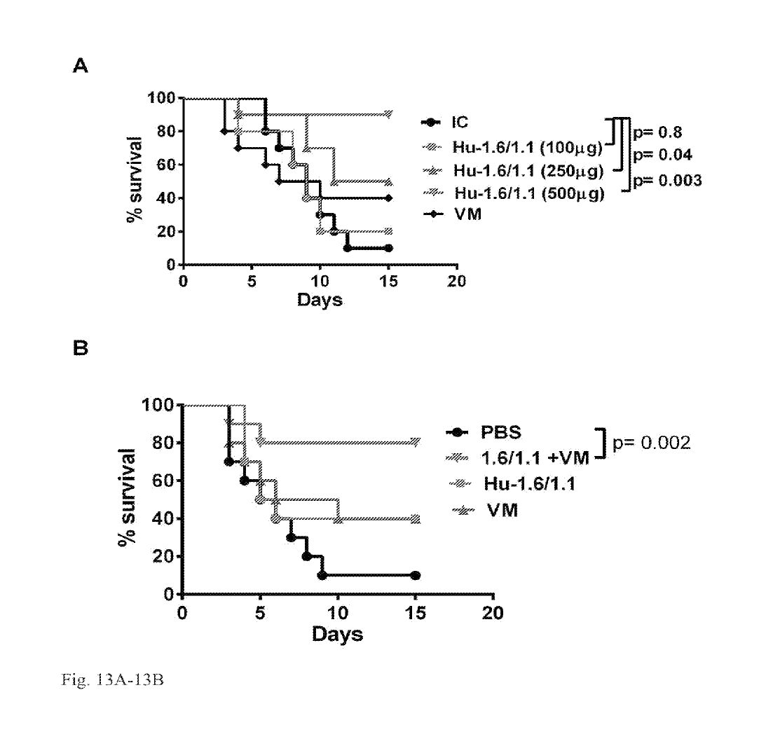

Treatment with Hu-mAb 1.6/1.1 enhances survival of mice with S. aureus sepsis: The protective efficacy of Hu-mAbs 1.6/1.1 and 1.4/1.1 was further explored using a murine S. aureus sepsis model (n=10 per group). These experiments demonstrated that i.v. treatment with Hu-mAb 1.6/1.1 (500 μg) in mice with S. aureus sepsis resulted in 90% survival (p=0.003) compared to 10% survival in mice treated with isotype control mAb (IC) (FIG. 13A). Survival benefit was demonstrated in a dose-dependent manner (FIG. 13A) and still appreciated at lower doses. Efficacy of Hu-1.4/1.1 was found to be less potent in this model (40% survival at dose of 500 μg compared with 90% survival at 500 μg of Hu-1.6/1.1 as shown on FIG. 13A).

Next, the efficacy of a lower dose of Hu-1.6/1.1, which by itself resulted in 50% survival, combined with vancomycin, which also has a similar efficacy when used alone, was investigated. These experiments demonstrated that combination treatment of Hu-1.6/1.1 (250 μg) with vancomycin significantly enhanced survival (to 90%) in mice with MRSA sepsis. In mice with MRSA sepsis that were treated with the combination (FIG. 13B), survival benefit was observed when compared to PBS treated mice (p=0.002), Vancomycin alone treated mice (p=0.08), as well as mice treated with 0Hu-1.6/1.1 (p=0.09) alone. Cytokine levels were measured in serum of mice on day 0, and 3 post infections.

As shown in Table 7, below, the two-way ANOVA of the dataset for IFN-γ showed a significant interaction between vancomycin and Hu-1.6/1.1 suggesting a synergistic effect. The one-way ANOVA showed a significant effect for both. The post hoc comparisons for IFN-γ showed the following groups to be significantly different (p<0.05): Hu-1.6/1.1, Hu-1.6/1.1 and vancomycin, Vancomycin only and PBS only. These data demonstrate a synergistic effect by the combination of the antibody of the invention and Vancomycin.

TABLE 7
Multiplex cytokine analysis of serum from mice treated with Hu-mAb
1.6/1.1, 1.6/1.1 + Vancomycin, Vancomycin alone or PBS and infected with 5 × 10⁷ CFU S. aureus
strain 38 i.v. Data are represented as mean +/− SE from 10 mice.

| | IFN-γ⁺ | IL-10^{‡} | IL-12p70^{‡} | IL-1β^{‡} | IL-6 | KC/GRO | TNF-α^{‡} |
|---|---|---|---|---|---|---|---|
| Hu-1.6/ | 2.26 ± 0.77* | 12.42 ± 1.61* | 46.77 ± 9.96* | 5.03 ± 1.74 | 433.50 ± 314.41 | 284.19 ± 88.71 | 1.71 ± 0.46 |
| 1.1 | | | | | | | |
| Hu-1.6/ | 1.66 ± 0.89* | 9.04 ± 2.76* | 42.88 ± 11.29* | 4.58 ± 3.97 | 384.27 ± 363.03 | 248.53 ± 167.89 | 1.41 ± 0.49* |
| 1.1 + VC | | | | | | | |
| VC | 2.98 ± 1.64 | 16.75 ± 6.67 | 61.55 ± 23.71* | 3.51 ± 1.08* | 379.72 ± 335.45 | 260.69 ± 122.01 | 1.46 ± 0.68 |
| PBS | 5.03 ± 1.33 | 17.26 ± 4.12 | 69.33 ± 28.54 | 5.87 ± 1.69 | 465.80 ± 255.05 | 292.77 ± 132.22 | 2.01 ± 0.56 |

⁺Two way Anova analyis showed ⁺synergistic effect of vancomycin and antibody in IFN-γ,
^{‡}additive effect of mAb in IL-10 and additive effect of vancomycin in IL-1, IL-10, IL-12 and TNF-α.
*p < 0.05, by t-test showing significantly lower levels of of IFN-γ, IL-10, IL-12p70 and TNF-α when compared to PBS treated mice.

### REFERENCES

1. Iandolo, J. J., and Shafer, W. M. (1977) Infect. Immun 16, 610-616
2. Swaminathan, S., Furey, W., Pletcher, J., and Sax, M. (1992) Nature 359, 801-806
3. Kappler, J. W., Herman, A., Clements, J., and Marrack, P. (1992) J. Exp. Med. 175, 387-396
4. Faulkner, L., Cooper, A., Fantino, C., Altmann, D. M., and Sriskandan, S. (2005) J. Immunol. 175, 6870-6877
5. Miethke, T., Wahl, C., Heeg, K., Echtenacher, B., Krammer, P. H., and Wagner, H. (1992) J. Exp. Med. 175, 91-98
6. Bachert, C., Zhang, N., Patou, J., van Zele, T., and Gevaert, P. (2008) Curr. Opin. Allergy Clin. Immunol. 8, 34-38
7. Liu, T., Kong, W., Yang, P., and Wang, B. (2006) J. Huazhong. Univ. Sci. Technolog. Med. Sci. 26, 63-67
8. Rossi, R. E., and Monasterolo, G. (2004) Int. Arch. Allergy Immunol. 133, 261-266
9. Rossi, R. J., Muralimohan, G., Maxwell, J. R., and Vella, A. T. (2004) Int. Immunol. 16, 1751-1760
10. Ulrich, R. G., Sidell, S., Taylor, T. J., Wilhelmsen, C. L., and Franz, D. R. (2001) Textbook of Military Medicine: Medical Aspects of Chemical and Biological Warfare, Borden Institute, Washington D.C.
11. Darenberg, J., So″derquist, B., Normark, B. H., and Norrby-Teglund, A. (2004) Clin Infect. Dis. 38, 836-842

1. An isolated antibody that specifically binds to a staphylococcal enterotoxin B (SEB) and comprises (A) (i) a heavy chain variable region (VH) comprising a VH complementarity determining region (CDR) one (CDR1) comprising SEQ ID NO:9, a VH CDR2 comprising SEQ ID NO:10, and VH CDR3 comprising SEQ ID NO:11; or (ii) a light chain variable region (VL) comprising a VL CDR1 comprising SEQ ID NO:12, a VL CDR2 comprising SEQ ID NO:13, and a VL CDR3 comprising SEQ ID NO:14, or (iii) a heavy chain variable region (VH) comprising a VH CDR1 comprising SEQ ID NO:9, a VH CDR2 comprising SEQ ID NO:10, a VH CDR3 comprising SEQ ID NO:11, a VL CDR1 comprising SEQ ID NO:12, a VL CDR2 comprising SEQ ID NO:13, and a VL CDR3 comprising SEQ ID NO:14,
and (B) an Fc portion having a sequence at least 90% identical to a human Fc region and/or a variable domain framework sequence having a sequence at least 90% identical to a human variable domain framework sequence,
or an SEB-binding fragment of such antibody.

2. The isolated antibody or SEB-binding antibody fragment of claim 1, comprising a variable domain framework sequence having a sequence at least 90% identical to a human variable domain FR1, FR2, FR3 or FR4.

3. The isolated antibody or SEB-binding antibody fragment of claim 1, comprising the VH sequence of SEQ ID NO:15, 19, 21, 23, or 25 or having a sequence at least 90% identical thereto.

4. The isolated antibody or SEB-binding antibody fragment of claim 1, comprising the VL sequence of SEQ ID NO:16, 27, or 29 or having a sequence at least 90% identical thereto.

5. The isolated antibody or SEB-binding antibody fragment of claim 1, wherein the VH comprises a VH CDR1 comprising SEQ ID NO:9, a VH CDR2 comprising SEQ ID NO:10, and a VH CDR3 comprising SEQ ID NO:11, and wherein the VL comprises a VL CDR1 comprising SEQ ID NO:12, a VL CDR2 comprising SEQ ID NO:13, and a VL CDR3 comprising SEQ ID NO:14.

6. The isolated antibody or SEB-binding antibody fragment of claim 1, wherein the variable domain framework of the VH comprises (a) the amino acid sequence of SEQ ID NO: 15, 19, 21, 23, or 25 or (b) a variant of one of SEQ ID NOS: 15, 19, 21, 23, or 25 with one to five conservative amino acid substitutions in residues that are not within a CDR1, CDR2 or CDR3 thereof.

7. The isolated antibody or SEB-binding antibody fragment of claim 1, wherein the variable domain framework of the VL comprises (a) the amino acid sequence of SEQ ID NO: 16, 27 or 29, or (b) a variant of one of SEQ ID NOS: 16, 27 or 29, with one to five conservative amino acid substitutions in residues that are not within a CDR1, CDR2 or CDR3 thereof.

8. The isolated antibody or SEB-binding antibody fragment of claim 1, comprising an Fc region having a sequence identical to a human Fc region.

9. (canceled)

10. The isolated antibody or SEB-binding antibody fragment of claim 1, comprising a variable domain framework sequence having a sequence identical to a human variable domain framework sequence FR1, FR2, FR3 or FR4.

11. The isolated antibody or SEB-binding antibody fragment of claim 1, wherein the antibody has an isotype that is selected from the group consisting of IgG₂, IgG_{2Δa}, IgG₄, IgG_{4Δb}, IgG_{4Δc}, IgG₄ S228P, IgG_{4Δb} S228P and IgG_{4Δc} S228P.

12. (canceled)

13. The isolated antibody of claim 1.

14. The SEB-binding antibody fragment of claim 1.

15. A single-chain variable fragment (scFv) that (a) specifically binds to a staphylococcal enterotoxin B (SEB) and comprises a heavy chain variable region (VH) and a light chain variable region (VL) linked to each other by a linker peptide, wherein (i) the VH comprises a VH CDR1 comprising SEQ ID NO:9, a VH CDR2 comprising SEQ ID NO:10, a VH CDR3 comprising SEQ ID NO:11, or (ii) the VL comprises a VL CDR1 comprising SEQ ID NO:12, a VL CDR2 comprising SEQ ID NO:13, and a VL CDR3 comprising SEQ ID NO:14; or (iii) the VH comprises a VH CDR1 comprising SEQ ID NO:9, a VH CDR2 comprising SEQ ID NO:10, a VH CDR3 comprising SEQ ID NO:11, and the VL comprises a VL CDR1 comprising SEQ ID NO:12, a VL CDR2 comprising SEQ ID NO:13, and a VL CDR3 comprising SEQ ID NO:14, and (b) comprises a variable domain framework sequence thereof has a sequence at least 90% identical to a human variable domain framework sequence.

16-23. (canceled)

24. An isolated antibody or antigen-binding portion thereof, comprising an amino acid sequence encoded by an insert present in (i) plasmid hu20B1 VL v1.1 deposited with the ATCC and having ATCC Accession No. PTA-13616; (ii) plasmid hu20B1 VH v1.4 deposited with the ATCC and having ATCC Accession No. PTA-13617; or (iii) plasmid hu20B1 VH v1.6 deposited with the ATCC and having ATCC Accession No. PTA-13618.

25-29. (canceled)

30. A method of treating a subject having, or at risk for, a staphylococcal disease, the method comprising administering to an subject having or at risk for a staphylococcal disease an amount of the antibody or fragment of claim 1 effective to treat a subject having, or at risk for, a staphylococcal disease.

31. A method of treating a subject having, or at risk for, a staphylococcal disease, the method comprising administering to an subject having or at risk for a staphylococcal disease an amount of the scFv of claim 1 effective to treat a subject having, or at risk for, a staphylococcal disease.

32. (canceled)

46. A pharmaceutical composition comprising an antibody or antigen-binding fragment thereof, or scFv, of claim 1, and a pharmaceutically acceptable carrier.

47. The pharmaceutical composition of claim 46, further comprising a therapeutically effective amount of Vancomycin.

48. The pharmaceutical composition of claim 46, further comprising an amount of Vancomycin, wherein the combined amount of antibody, or antigen-binding fragment thereof, or scFv, and Vancomycin is a therapeutically effective synergistic amount.
